(19) United States
(12) Patent Application Publication
(10) Pub. No.: US 2020/0325139 A1
(43) Pub. Date: Oct. 15, 2020

(54) NEW ANTIBIOTICS TARGETING MYCOBACTERIA

(71) Applicants: ASSISTANCE PUBLIQUE-HOPITAUX DE PARIS, Paris (FR); UNIVERSITE DE PARIS, Paris (FR); SORBONNE UNIVERSITE, Paris (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR)

(72) Inventors: Jean-Luc MAINARDI, Viroflay (FR); Michel ARTHUR, Arcueil (FR); Mélanie ETHEVE-QUELQUEJEU, Choisy le Roi (FR); Laura IANNAZZO, Gournay sur Marne (FR)

(21) Appl. No.: 16/956,300

(22) PCT Filed: Dec. 21, 2018
(86) PCT No.: PCT/EP2018/086807

§ 371 Date: Jun. 19, 2020

(30) Foreign Application Priority Data
Dec. 22, 2017 (EP) 17306902.2

(51) Int. Cl. C07D 471/08; A61K 31/5377; C07F 7/08; A61K 31/439; A61K 31/695; A61P 31/04; A61P 31/06; A61K 31/43

(52) U.S. Cl. CPC C07D 471/08; A61K 31/5377; C07F 7/0814; A61K 31/43; A61K 31/695; A61P 31/04; A61P 31/06; A61K 31/439

(57) ABSTRACT

The present invention relates to a compound of the folowing formula (I) or a pharmaceutically acceptable salt and/or solvate thereof, notably for use as a dmg, notably in the treatment of a disease caused by mycobacteria, as well as pharmaceutical compositions containing such a compound and a process to prepare such a compound.

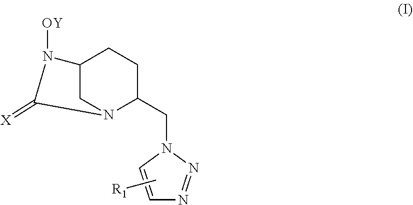

The present invention relates to new diazabicyclooctane (DBO) derivatives, in particular for their use as a drug, notably in the treatment of a disease caused by mycobacteria, synthetic procedures for preparing them and pharmaceutical compositions containing such compounds.

Tuberculosis is the second infectious disease leading to mortality after AIDS and one of the top ten causes of death worldwide. The emergence of multidrug-resistant strains had complicated the management of tuberculosis and constitutes a serious threat for the control of the pandemic. Drugs of the β-lactam family have regained interest for the treatment of tuberculosis since the β-lactamase produced by Mycobacterium tuberculosis (BlaC) is irreversibly inactivated by clavulanate [Hugonnet et al. Science 2009, 323, 1215-1218]. The targets of β-lactams are unusual in M. tuberculosis since the cross-linking step of cell wall peptidoglycan synthesis is mainly (80%) performed by L,D-transpeptidases (LDTs) instead of the classical D,D-transpeptidases belonging to the penicillin-binding protein (PBP) family [Lavollay et al. J. Bacteriol. 2008, 190, 4360-4366; Gupta et al. Nat. Med. 2010, 16, 466-469]. β-lactams of the carbapenem class, such as meropenem, are active against M. tuberculosis LDTs and this drug has recently shown efficacy in combination with clavulanate in a phase II clinical trial [Diacon et al. N. Engl. J. Med. 2016, 375, 393-394]. However, there are several limitations to the use of carbapenems for the treatment of tuberculosis including their broad antibacterial spectra, which result in adverse effects on the commensal flora, leading to opportunistic fungal infections and selection of broad-spectrum β-lactamases in the enterobacteria. Mycobacterium abscessus, a fast-growing mycobacterium, raises distinct medical issues. This bacterium has emerged in recent years as an important opportunistic pathogen increasingly responsible for mortality in cystic fibrosis patients and in the context of chronic obstructive pulmonary diseases [Floto et al. Thorax 2016, 71 Suppl 1:i1-i22]. The carbapenem imipenem is part of the recommended treatment of lung infections due to M. abscessus but the efficacy of this drug is limited by a broad β-lactamase (Bla_{Mab}), which is not inhibited by clavulanate [Dubee et al. J. Antimicrob. Chemother. 2015, 70, 1051-1058]. As for M. tuberculosis, the peptidoglycan of M. abscessus is mainly cross-linked by LDTs [Lavollay et al. J. Bacteriol. 2011, 193, 778-782].

There exists thus a need for new antibiotics targeting mycobacteria also resistant to hydrolysis by the β-lactamase Bla_{Mab} and BlaC in order to avoid the need for the association with a β-lactamase inhibitor.

Avibactam, a new β-lactamase inhibitor, has recently obtained regulatory approval in the USA and Europe [Papp-Wallace et al. Infect. Dis. Clin. North. Am. 2016, 30, 441-464]. Avibactam is original both in its mode of action and its structure since it is based on a diazabicyclooctane (DBO) scaffold containing a five-membered ring. It reversibly inactivates β-lactamases containing an active-site serine by formation of a carbamoyl-enzyme, which is not prone to hydrolysis.

By functionalizing the DBO scaffold, the inventors have developed new antibiotics targeting mycobacteria.

The present invention thus relates to a compound of the following general formula (I):

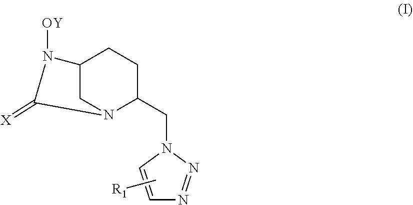

or a pharmaceutically acceptable salt and/or solvate thereof, wherein:

X is O or S;

Y is SO₃H or PO₃H; and

R₁ is:
H,
a tri-(C₁-C₆)alkylsilyl group,
a (C₁-C₆)alkyl group, optionally substituted with one or several groups selected from halo, cyano (CN), OR₂, SR₃, NR₄R₅, COR₆, CO₂R₇, CONR₈R₉ and NO₂, or
an aryl, heteroaryl, aryl-(C₁-C₆)alkyl, heteroaryl-(C₁-C₆)alkyl, cycloalkyl, cycloalkyl-(C₁-C₆)alkyl, heterocycle or heterocycle-(C₁-C₆)alkyl group, optionally substituted with one or several groups selected from halo, cyano (CN), (C₁-C₆)alkyl, OR₁₀, SR₁₁, NR₁₂R₁₃, COR₁₄, CO₂R₁₅, CONR₁₆R₁₇ and NO₂,
   wherein R₂ to R₁₇ are, independently of each other, H, a (C₁-C₆)alkyl group or a C(═O)O₁-C₆)alkyl.

In a preferred embodiment, the present invention relates to a compound of the following general formula (I):

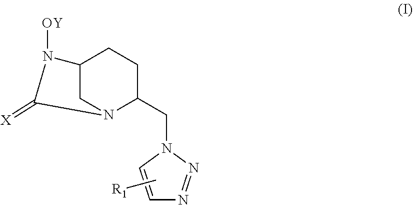

or a pharmaceutically acceptable salt and/or solvate thereof, wherein:

X is O or S;

Y is SO₃H or PO₃H; and

R₁ is:
a tri-(C₁-C₆)alkylsilyl group,
a (C₁-C₆)alkyl group, optionally substituted with one or several groups selected from halo, cyano (CN), OR₂, SR₃, NR₄R₅, COR₆, CO₂R₇, CONR₈R₉and NO₂, or
an aryl, heteroaryl, aryl-(C₁-C₆)alkyl, heteroaryl-(C₁-C₆)alkyl, cycloalkyl, cycloalkyl-(C₁-C₆)alkyl, heterocycle or heterocycle-(C₁-C₆)alkyl group, optionally substituted with one or several groups selected from halo, cyano (CN), (C₁-C₆)alkyl, OR₁₀, SR₁₁, NR₁₂R₁₃, COR₁₄, CO₂R₁₅, CONR₁₆R₁₇ and NO₂,
   wherein R₂ to R₁₇ are, independently of each other, H or a (C₁-C₆)alkyl group.

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt and/or solvate” is intended to mean, in the framework of the present invention, a salt and/or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

In particular, a pharmaceutically acceptable salt of a compound of the invention is a sodium salt.

Acceptable solvates for the therapeutic use of the compounds of the present invention include conventional solvates such as those formed during the last step of the preparation of the compounds of the invention due to the presence of solvents. As an example, mention may be made of solvates due to the presence of water (these solvates are also called hydrates) or ethanol.

The term “halo”, as used in the present invention, refers to bromo, chloro, iodo or fluoro.

The term “(C₁-C₆)alkyl”, as used in the present invention, refers to a straight or branched saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “(C₁-C₃)alkyl”, as used in the present invention, refers to a straight or branched saturated hydrocarbon chain containing from 1 to 3 carbon atoms in particular to methyl, ethyl, n-propyl and iso-propyl.

The term “tri-(C₁-C₆)alkylsilyl”, as used in the present invention, refers to a group of formula —SiAlk₁Alk₂Alk₃ with Alk₁, Alk₂ and Alk₃ each representing independently a (C₁-C₆)alkyl group as defined above. It can be for example trimethylsilyl, triethylsilyl, t-butyldimethylsilyl and the like.

The term “cycloalkyl” as used in the present invention refers to a saturated hydrocarbon ring comprising from 3 to 7, advantageously from 5 to 7, carbon atoms including, but not limited to, cyclohexyl, cyclopentyl, cyclopropyl, cycloheptyl and the like.

The term “cycloalkyl-(C₁-C₆)alkyl” as used in the present invention refers to any cycloalkyl group as defined above, which is bound to the molecule by means of a (C₁-C₆-alkyl group as defined above.

The term “aryl”, as used in the present invention, refers to an aromatic hydrocarbon group comprising preferably 6 to 10 carbon atoms and comprising one or more fused rings, such as, for example, a phenyl or naphtyl group. Advantageously, it is a phenyl group.

The term “aryl-(C₁-C₆)alkyl”, as used in the present invention, refers to an aryl group as defined above bound to the molecule via a (C₁-C₁₀alkyl group as defined above. In particular, it is a benzyl group.

The term “heterocycle” as used in the present invention refers to a saturated or unsaturated non-aromatic monocycle or polycycle, comprising fused, bridged or spiro rings, preferably fused rings, advantageously comprising 3 to 10, notably 3 to 6, atoms in each ring, in which the atoms of the ring(s) comprise one or more, advantageously 1 to 3, heteroatoms selected from O, S and N, preferably O and N, the remainder being carbon atoms.

A saturated heterocycle is more particularly a 3-, 4-, 5- or 6-membered, even more particularly a 5- or 6-membered saturated monocyclic heterocycle such as an aziridine, an azetidine, a pyrrolidine, a tetrahydrofurane, a 1,3-dioxolane, a tetrahydrothiophene, a thiazolidine, an isothiazolidine, an oxazolidine, an isoxazolidine, an imidazolidine, a pyrazolidine, a triazolidine, a piperidine, a piperazine, a 1,4-dioxane, a morpholine or a thiomorpholine.

An unsaturated heterocycle is more particularly an unsaturated monocyclic or bicyclic heterocycle, each cycle comprising 5 or 6 members, such as 1H-azirine, a pyrroline, a dihydrofurane, a 1,3-dioxolene, a dihydrothiophene, a thiazoline, an isothiazoline, an oxazoline, an isoxazoline, an imidazoline, a pyrazoline, a triazoline, a dihydropyridine, a tetrahydropyridine, a dihydropyrimidine, a tetrahydropyrimidine, a dihydropyridazine, a tetrahydropyridazine, a dihydropyrazine, a tetrahydropyrazine, a dihydrotriazine, a tetrahydrotriazine, a 1,4-dioxene, an indoline, a 2,3-dihydrobenzofurane (coumaran), a 2,3-dihydrobenzothiophene, a 1,3-benzodioxole, a 1,3-benzoxathiole, a benzoxazoline, a benzothiazoline, a benzimidazoline, a chromane or a chromene.

The term “heterocycle-(C₁-C₆alkyl” as used in the present invention refers to a heterocycle group as defined above, which is bound to the molecule by means of a (C₁-C₆-alkyl group as defined above.

The term “heteroaryl” as used in the present invention refers to an aromatic heterocycle as defined above. It can be more particularly an aromatic monocyclic or bicyclic heterocycle, each cycle comprising 5 or 6 members, such as a pyrrole, a furane, a thiophene, a thiazole, an isothiazole, an oxazole, an isoxazole, an imidazole, a pyrazole, a triazole, a pyridine, a pyrimidine, an indole, a benzofurane, a benzothiophene, a benzothiazole, a benzoxazole, a benzimidazole, an indazole, a benzotriazole, a quinoline, an isoquinoline, a cinnoline, a quinazoline or a quinoxaline.

The term “heteroaryl-(C₁-C₆)alkyl” as used in the present invention refers to a heteroaryl group as defined above, which is bound to the molecule by means of a (C₁-C₆)-alkyl group as defined above.

The stereoisomers of the compounds of general formula (I) also form part of the present invention, as well as the mixtures thereof, in particular in the form of a racemic mixture.

The tautomers of the compounds of general formula (I) also form part of the present invention.

Within the meaning of this invention, “stereoisomers” is intended to designate configurational isomers, notably diastereoisomers or enantiomers. The configurational isomers result from different spatial position of the substituents on a carbon atom comprising four different substituents. This atom thus constitutes a chiral or asymmetric center. Configurational isomers that are not mirror images of one another are designated as “diastereoisomers,” and configurational isomers that are non-superimposable mirror images are designated as “enantiomers”.

An equimolar mixture of two enantiomers of a chiral compound is designated as racemate or racemic mixture.

By “tautomer” is meant, within the meaning of the present invention, a constitutional isomer of the compound obtained by prototropy, i.e. by migration of a hydrogen atom and concomitant change of location of a double bond. The different tautomers of a compound are generally interconvertible and present in equilibrium in solution, in proportions that can vary according to the solvent used, the temperature or the pH.

A compound according to the invention corresponds to one of the constitutional isomers of the following general formulas (Ia) and (Ib):

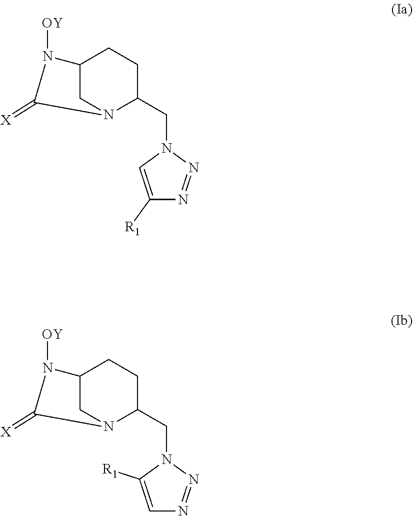

A compound of formula (Ia) may correspond to one of the stereoisomers of the following general formulas (Ia.i), (Ia.ii), (Ia.iii) and (Ia.iv):

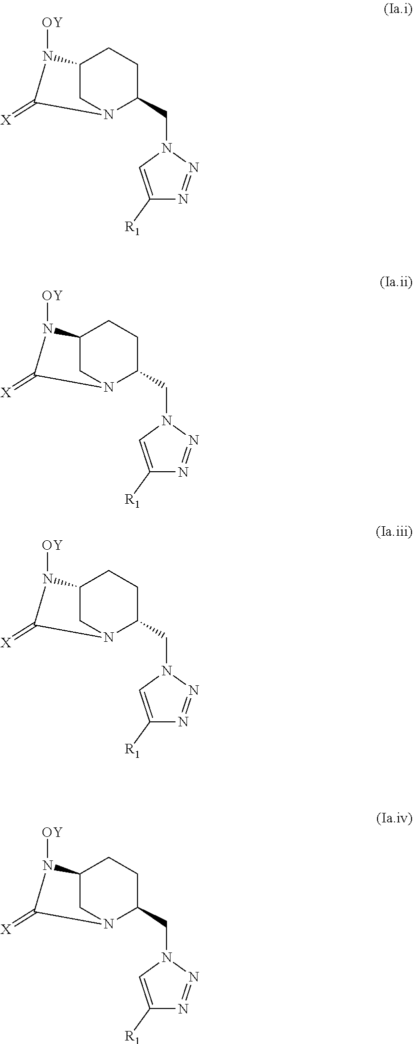

A compound of formula (Ib) may correspond to one of the stereoisomers of the following general formulas (Ib.i), (Ib.ii), (Ib.iii) and (Ib.iv):

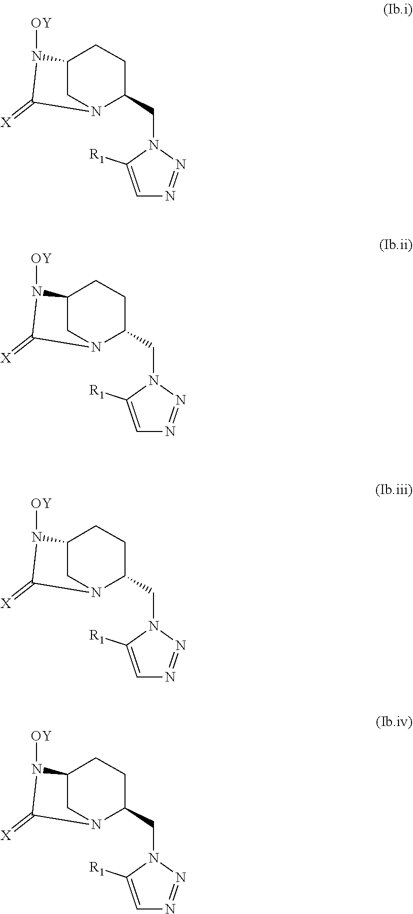

In a particular embodiment, X represents an oxygen atom.

In a particular embodiment, Y represents a SO₃H group.

In a particular embodiment, R₁ is:
a tri-(C₁-C₁)alkylsilyl group,
a (C₁-C₆)alkyl group, optionally substituted with one or several groups selected from halo, OR₂, NR₄R₅, CO₂R₇ and CONR₈R₉, or
an aryl, heteroaryl, aryl-(C₁-C₆)alkyl, heteroaryl-(C₁-C₆)alkyl, cycloalkyl, cycloalkyl-(C₁-C₆)alkyl, heterocycle or heterocycle-(C₁-C₆)alkyl group, optionally substituted with one or several groups selected from halo, (C₁-C₆)alkyl, OR₁₀, NR₁₂R₁₃, CO₂-R₁₅ and CONR₁₆R₁₇.

In another particular embodiment, R₁ is:
a tri-(C₁-C₆)alkylsilyl group,
a (C₁-C₆)alkyl group, optionally substituted with one or several groups selected from halo, OR₂, NR₄R₅, CO₂R₇ and CONR₈R₉, or
an aryl, heteroaryl, aryl-(C₁-C₆)alkyl, heteroaryl-(C₁-C₆)alkyl, heterocycle or heterocycle-(C₁-C₆)alkyl group, optionally substituted with one or several groups selected from halo, (C₁-C₆)alkyl, OR₁₀, NR₁₂R₁₃, CO₂R₁₅ and CONR₁₆R₁₇.

In still another particular embodiment, R₁ is:
a tri-(C₁-C₆)alkylsilyl group,
a (C₁-C₆)alkyl group, optionally substituted with one or several groups selected from halo, OR₂, NR₄R₅, CO₂R₇ and CONR₈R₉, or
an aryl, heteroaryl, aryl-(C₁-C₃)alkyl, heteroaryl-(C₁-C₃)alkyl, heterocycle or is heterocycle-(C₁-C₃)alkyl group, optionally substituted with one or several groups selected from halo, (C₁-C₃)alkyl, OR₁₀, NR₁₂R₁₃, CO₂R₁₅ and CONR₁₆R₁₇.

In yet another particular embodiment, R₁ is:
a tri-(C₁-C₆)alkylsilyl group,
a (C₁-C₆)alkyl group, optionally substituted with one or several groups selected from halo, OR₂, NR₄R₅, CO₂R₇ and CONR₈R₉, or
an aryl, heteroaryl or heterocycle-(C₁-C₃)alkyl group, optionally substituted with one or several groups selected from halo, (C₁-C₃)alkyl, OR₁₀, NR₁₂R₁₃, CO₂R₁₅ and CONR₁₆R₁₇.

In the above embodiments of R₁, the tri-(C₁-C₆)alkylsilyl group may be in particular selected in the group consisting of trimethylsilyl, triethylsilyl and t-butyldimethylsilyl; preferably, it is a trimethylsilyl group.

In the above embodiments of R₁:
the aryl moiety in the aryl, aryl-(C₁-C₆)alkyl and aryl-(C₁-C₃)alkyl groups may be preferably a phenyl;
the heteroaryl moiety in the heteroaryl, heteroaryl-(C₁-C₆)alkyl and heteroaryl-(C₁-C₃)alkyl groups may be in particular a 5- or 6-membered heteroaryl comprising one or two heteroatoms chosen from O and N, notably selected from furan, pyrrole, imidazole, pyridine, pyrazine and pyrimidine; preferably, it is a pyridine;
the heterocycle moiety in the heterocycle, heterocycle-(C₁-C₆)alkyl and heterocycle-(C₁-C₃)alkyl groups may be in particular a 5- or 6-membered, saturated or unsaturated, preferably saturated heterocycle comprising one or two heteroatoms chosen from O and N, notably selected from pyrrolidine, piperidine, morpholine and piperazine, preferably, it is a pyrrolidine or a piperidine optionally substituted by CO₂R₁₅;
the cycloalkyl moiety in the cycloalkyl and cycloalkyl-(C₁-C₆)alkyl groups may be in particular a cyclohexyl, cyclopentyl or cyclopropyl.

In the above embodiments of R₁, R₂ to R₁₇ may be, independently of each other, in particular H or a methyl, ethyl, n-propyl, iso-propyl group or iso-butyl group or C(═O)O(C₁-C₆)alkyl, preferably C(═O)OtBu, notably H

According to a particular embodiment, a compound of the invention is of general formula (I), wherein:

X is O;

Y is SO₃H; and

R₁ is:
a tri-(C₁-C₆)alkylsilyl group,
a (C₁-C₆)alkyl group, optionally substituted with one or several groups selected from halo, cyano (ON), OR₂, SR₃, NR₄R₅, COR₅, CO₂R₇, CONR₈R₉ and NO₂, or
an aryl, heteroaryl, aryl-(C₁-C₆)alkyl, heteroaryl-(C₁-C₆)alkyl, cycloalkyl, cycloalkyl-(C₁-C₆)alkyl, heterocycle or heterocycle-(C₁-C₆)alkyl group, optionally substituted with one or several groups selected from halo, cyano (ON), (C₁-C₆)alkyl, OR₁₀, SR₁₁, NR₁₂R₁₃, COR₁₄, CO₂R₁₅, CONR₁₆R₁₇ and NO₂,

R₂ to R17 being as defined above.

According to another particular embodiment:

X is O;

Y is SO₃H; and

R₁ is:
a tri-(C₁-C₆)alkylsilyl group, notably a trimethylsilyl group,
a (C₁-C₆)alkyl group, optionally substituted with one or several groups selected from halo, OR₂, NR₄R₅, CO₂R₇ and CONR₈R₉, or
an aryl, heteroaryl, aryl-(C₁-C₆)alkyl, heteroaryl-(C₁-C₆)alkyl, heterocycle or heterocycle-(C₁-C₆)alkyl group, optionally substituted with one or several groups selected from halo, (C₁-C₆)alkyl, OR₁₀, NR₁₂R₁₃, CO₂R₁₅ and CONR₁₆R₁₇;
wherein:
   the aryl moiety in the aryl and aryl-(C₁-C₆)alkyl groups is a phenyl;
   the heteroaryl moiety in the heteroaryl and heteroaryl-(C₁-C₆)alkyl groups is a 5- or 6-membered heteroaryl comprising one or two heteroatoms chosen from O and N, notably selected from furan, pyrrole, imidazole, pyridine, pyrazine and pyrimidine; preferably, it is a pyridine;
   the heterocycle moiety in the heterocycle and heterocycle-(C₁-C₆)alkyl groups is a 5- or 6-membered, saturated or unsaturated, preferably saturated heterocycle comprising one or two heteroatoms chosen from O and N, notably selected from pyrrolidine, piperidine, morpholine and piperazine, preferably, it is a pyrrolidine or a piperidine.

In a preferred embodiment, a compound of the invention is of general formula (Ia), wherein X, Y and R₁ are as defined above.

Notably, it is the stereoisomer of general formula (Ia.i).

In a particular embodiment, a compound of the present invention is chosen among the following compounds:

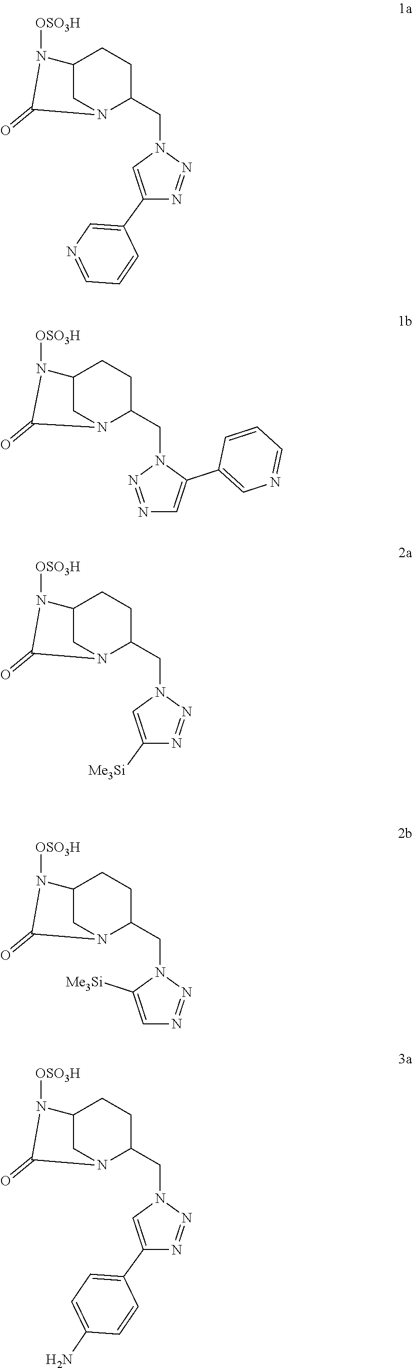

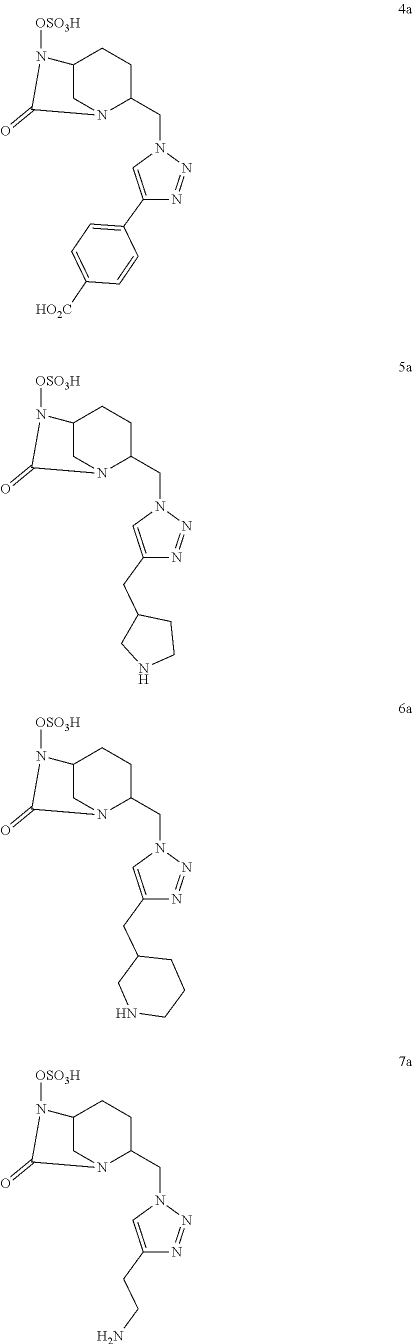

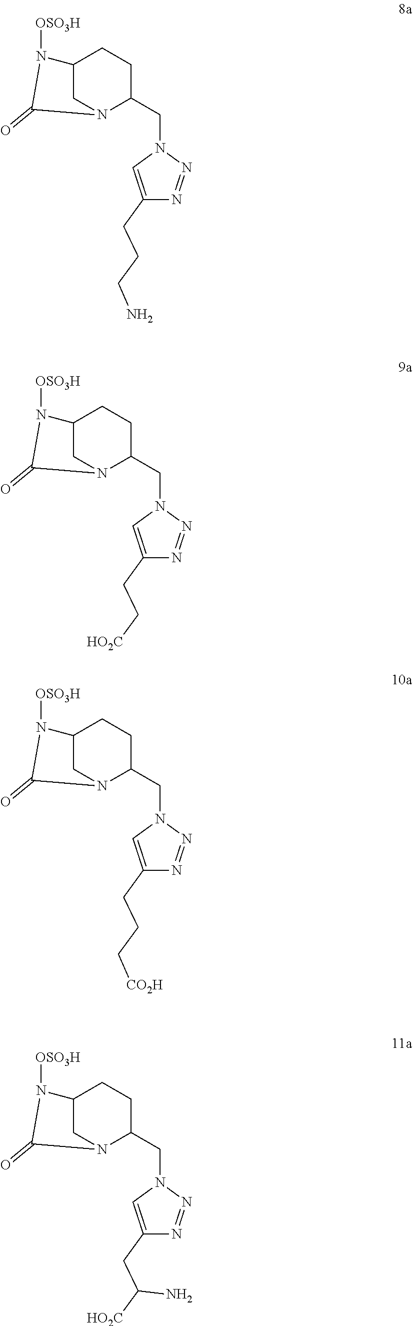

and the pharmaceutically acceptable salts, notably the sodium salts, and/or solvates thereof.

Notably, a compound of the present invention is chosen among the following compounds:

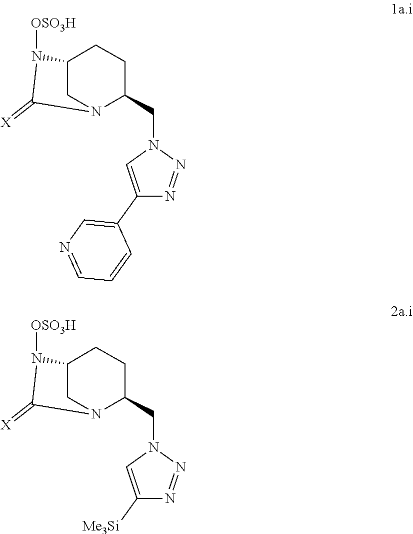

and the pharmaceutically acceptable salts, in particular the sodium salts, and/or solvates thereof.

In another particular embodiment, a compound of the present invention is chosen among the following compounds:

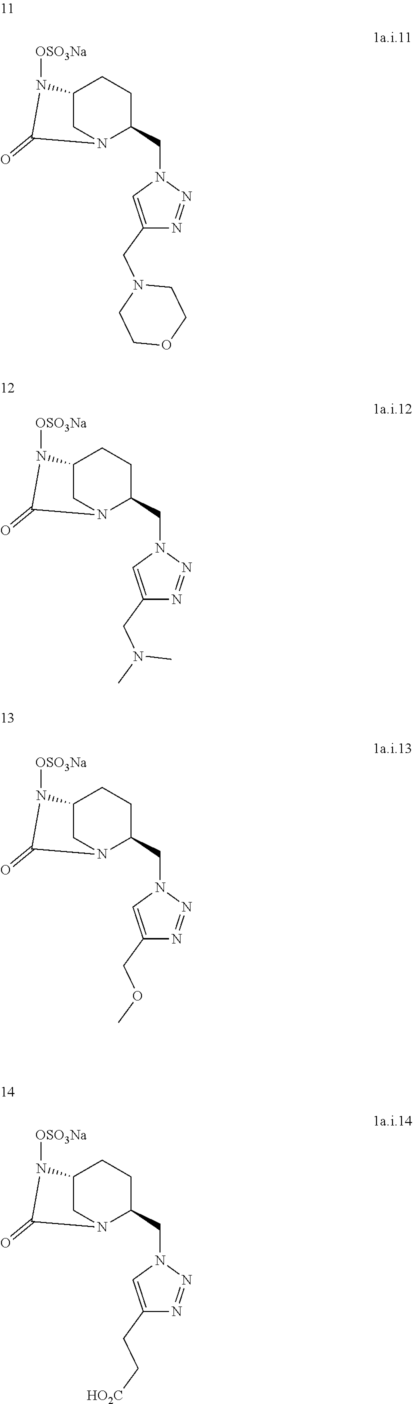

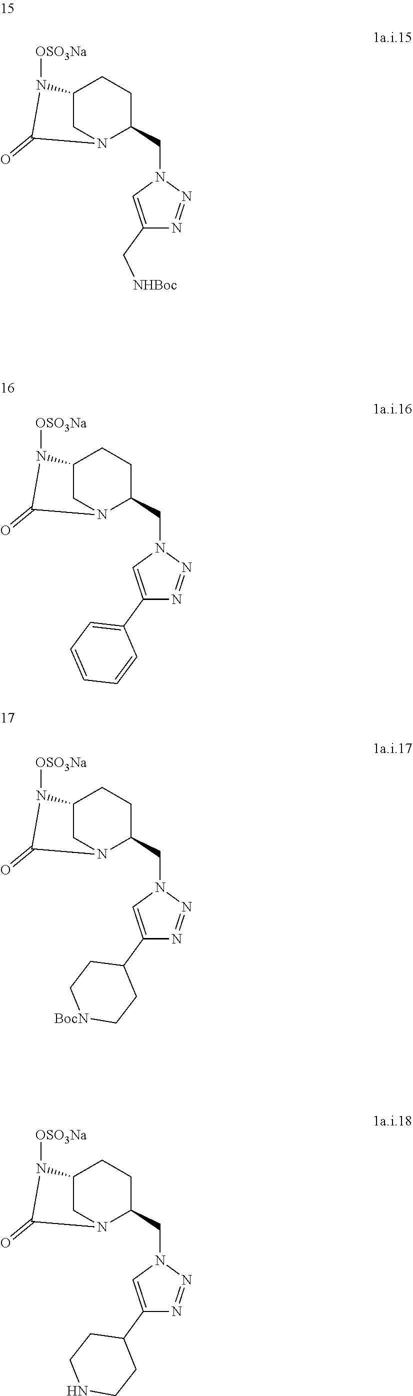

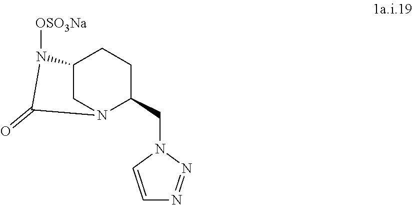

and the pharmaceutically acceptable salts, in particular the sodium salts, and/or solvates thereof.

The present invention also relates to a compound of formula (I) as defined previously for use as a β-lactamase inhibitor, L,D-transpeptidase inhibitor and/or D,D-transpeptidase inhibitor, notably for use as a β-lactamase inhibitor.

The present invention relates also to a compound of formula (I) as defined previously for use as a drug, notably intended for the treatment of a disease caused by mycobacteria.

The present invention concerns also the use of a compound of formula (I) as defined previously for the manufacture of a drug, notably intended for the treatment of a disease caused by mycobacteria.

The present invention concerns also a method for treating a disease caused by mycobacteria comprising the administration to a person in need thereof of an effective amount of a compound of formula (I) as defined previously.

The mycobacteria can be more particularly M. tuberculosis or M. abscessus.

The disease caused by mycobacteria, notably by M. tuberculosis or M. abscessus, may be in particular tuberculosis, lung infections in patients suffering from cystic fibrosis or a chronic obstructive pulmonary disease.

The present invention relates also to a pharmaceutical composition comprising at least one compound of formula (I) as defined previously and at least one pharmaceutically acceptable excipient.

The active principle can be administered in unitary dosage forms, in mixture with conventional pharmaceutical carriers, to animals and humans.

The pharmaceutical compositions according to the present invention are more particularly intended to be administered orally or parenterally (for ex. intravenously), notably to mammals including human beings.

Suitable unit dosage forms for administration comprise the forms for oral administration, such as tablets, gelatin capsules, powders, granules and oral solutions or suspensions.

When a solid composition is prepared in the form of tablets, the main active ingredient is mixed with a pharmaceutical vehicle such as gelatin, starch, lactose, magnesium stearate, talc, gum arabic and the like. The tablets may be coated with sucrose or with other suitable materials, or they may be treated in such a way that they have a prolonged or delayed activity and they continuously release a predetermined amount of active principle.

A preparation in gelatin capsules is obtained by mixing the active ingredient with a diluent and pouring the mixture obtained into soft or hard gelatin capsules.

A preparation in the form of a syrup or an elixir may contain the active ingredient together with a sweetener, an antiseptic, or also a taste enhancer or a suitable coloring agent.

The water-dispersible powders or granules may contain the active ingredient mixed with dispersing agents or wetting agents, or suspending agents, and with flavor correctors or sweeteners.

For parenteral administration, aqueous suspensions, isotonic saline solutions or sterile and injectable solutions which contain pharmacologically compatible dispersing agents and/or wetting agents are used.

The active principle may also be formulated in the form of microcapsules, optionally with one or more carrier additives.

The compounds of the invention can be used in a pharmaceutical composition at a dose ranging from 0.01 mg to 1000 mg a day, administered in only one dose once a day or in several doses along the day, for example twice a day. The daily administered dose is advantageously comprised between 5 mg and 500 mg, and more advantageously between 10 mg and 200 mg. However, it can be necessary to use doses out of these ranges, which could be noticed by the person skilled in the art.

The pharmaceutical compositions according to the present invention can comprise further at least another active principle, such as an antibiotic, notably a β-lactam antibiotic.

The present invention relates also to a pharmaceutical composition comprising:

(i) at least one compound of formula (I) as defined previously, and

(ii) at least another active principle, such as an antibiotic, notably a β-lactam antibiotic,

as a combination product for a simultaneous, separate or sequential use.

The β-lactam antibiotic may be in particular a member of the carbapenem class, such as meropenem or imipenem; a member of the penam (penicillin) class, such as amoxicillin; or a member of the cephem (cephalosporin) class, such as ceftriaxone or ceftaroline.

The present invention relates also to a pharmaceutical composition as defined previously for use in the treatment of a disease caused by mycobacteria.

The present invention concerns also a method for treating a disease caused by mycobacteria comprising the administration to a person in need thereof of an effective amount of a pharmaceutical composition according to the invention.

The present invention relates also to a process to prepare a compound of formula (I) as defined previously comprising a reaction converting the OH group of a compound of the following formula (II) into a OY group to obtain the corresponding compound of formula (I):

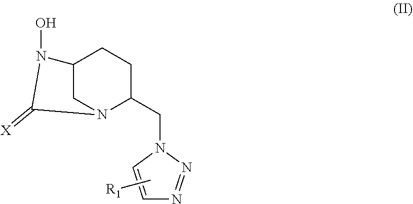

wherein X is O or S, and R₁ is as defined in claim 1, R₁ being optionally in a protected form,
wherein:
when Y is SO₃H, said reaction is a sulfonation reaction, and
when Y is PO₃H, said reaction is a phosphorylation reaction, followed by a deprotection of the R₁ group when it is in a protected form, optionally followed by a salt-forming step.

Sulfonation and phosphorylation reactions may be carried out under various reaction conditions that are well known to the one skilled in the art.

The optional deprotection and salt-forming steps and their reaction conditions are also well known to the skilled person.

The compound of formula (II) may be obtained in particular by a coupling reaction between:
a compound of the following formula (III):

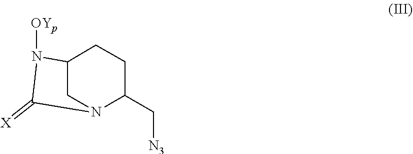

wherein X is O or S, and Yₚ is a hydroxyl protecting group, such as a benzyl group, and
a compound of the following formula (IV):

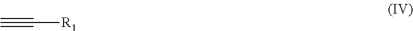

wherein R₁ is as defined in claim 1, optionally in a protected form,
followed by a deprotection of the OYₚ group.

Such a coupling reaction between an azide function (—N₃) and an alkyne function to obtain a 1,2,3-triazole is a well-known Click chemistry reaction, also called azide-alkyne Huisgen cycloaddition.

The azide-alkyne Huisgen reaction is usually catalysed by a copper (I) catalyst such as CuBr or CuI. The copper (I) catalyst can also be formed in situ by reduction of a copper (II) species, in particular by reduction of a copper (II) salt such as CuSO₄ in the presence of a reducing agent such as ascorbic acid or a salt thereof.

The Cu(I) catalysed 1,3-dipolar cycloaddition in between the azide and alkyne functions is regioselective. Indeed, the 1,4-triazole (IIa) is obtained as the sole product:

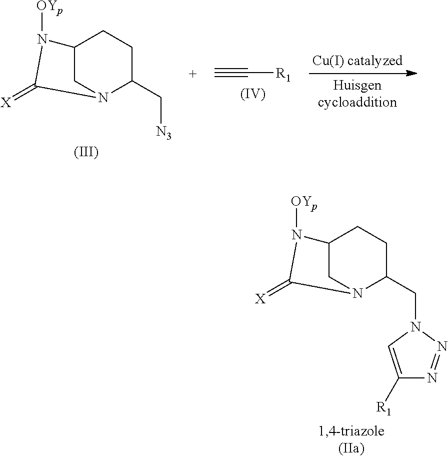

The cycloaddition can be performed in various solvents, such as tetrahydrofuran (THF), alcohols, dimethylsulfoxyde (DMSO), N,N-dimethylformamide (DMF), acetone, water or mixtures thereof.

The deprotection of the OYₚ group of a compound of formula (IIa) followed by a reaction converting the resulting OH group into a OY group allows the corresponding compound of formula (Ia).

The 1,5-regioisomer (IIb) may be obtained by a variant of the azide-alkyne coupling reaction using a Ru(II) catalyst, notably CpRuCl(PPh₃)₂, which is also regioslective [Zhang et. al. J. Am. Chem. Soc. 2005, 127(46), 15998-15999]:

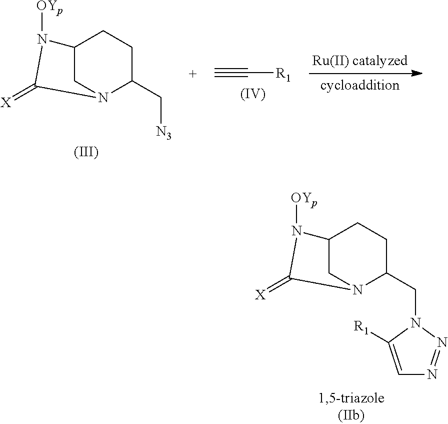

The deprotection of the OYₚ group of a compound of formula (11b) followed by a reaction converting the resulting OH group into a OY group allows the corresponding compound of formula (Ib).

A compound of formula (III) may correspond to one of the stereoisomers of the following general formulas (III.i), (III.ii), (III.iii) and (III.iv):

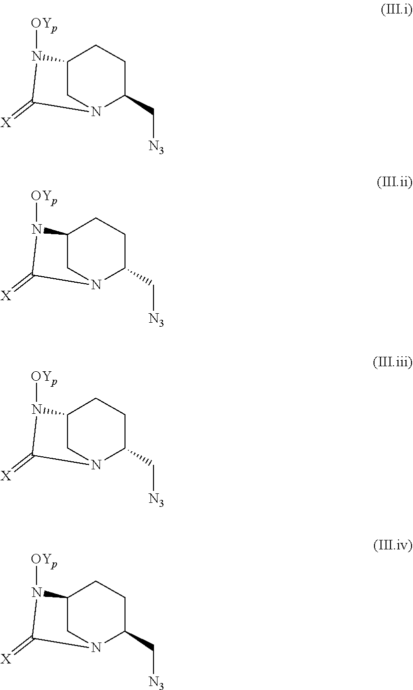

Said stereoisomers can notably be obtained by carrying out the methods detailed below in the examples.

The compound(s) obtained during the process described above can be separated from the reaction medium by methods well known to the person skilled in the art, such as by extraction, evaporation of the solvent or by precipitation or crystallisation (followed by filtration).

The compound(s) also can be purified if necessary by methods well known to the person skilled in the art, such as by recrystallisation, by distillation, by chromatography on a column of silica gel or by high performance liquid chromatography (HPLC).

The examples that follow illustrate the invention without limiting its scope in any way.

### EXAMPLES

### I. Synthesis of the Compounds According to the Invention

The following abbreviations are used in the following examples:
BOC=tert-butoxycarbonyl
b=broad
COD=cyclooctadiene
d=doublet
DBO=diazabicyclooctane
DCE=1,2-dichloroethene
DCM=dichloromethane
DEAD=diethyl azodicarboxylate
DIPEA=N,N-diisopropylethylamine
DMAP=4-dimethylaminopyridine
DMF=N,N-dimethylformamide
DMSO=N,N-dimethylsulfoxide
g=gram
h or hr=hour
HRMS=High resolution mass spectrometry
HPLC=High Performance Liquid Chromatography
Hz=Hertz
J=coupling constant
m=multiplet
M=Molar
M+H⁺=parent mass spectrum peak plus H⁺
mg=milligram
MIC=minimum inhibitory concentration
mL=milliliter
mM=millimolar
mmol=millimole

MS=mass spectrum
nM=nanomolar
NMR=Nuclear Magnetic Resonance
Ns=nitrosulfonyl
Pd/C=Palladium on charcoal
Pyr=pyridine
ppm=part per million
quant.=quantitative
RT=room temperature
s=singlet
sat.=saturated
t=triplet
TBAF=Tetrabutylammonium fluoride
TBDMS=tert-butyl-dimethyl-silyl
TEA=triethylamine
TFA=trifluoroacetic acid
THF=tetrahydrofuran
TLC=thin layer chromatography
μL=microliter
μM=micromolar

### I-1. Synthesis of the Intermediate Compounds of General Formula (Ill)

i) Stereoisomer (III.i)

The compound of formula III.i, wherein X is an oxygen atom and Yₚ is a benzyl group (compound 13) was prepared by carrying out the following successive steps:

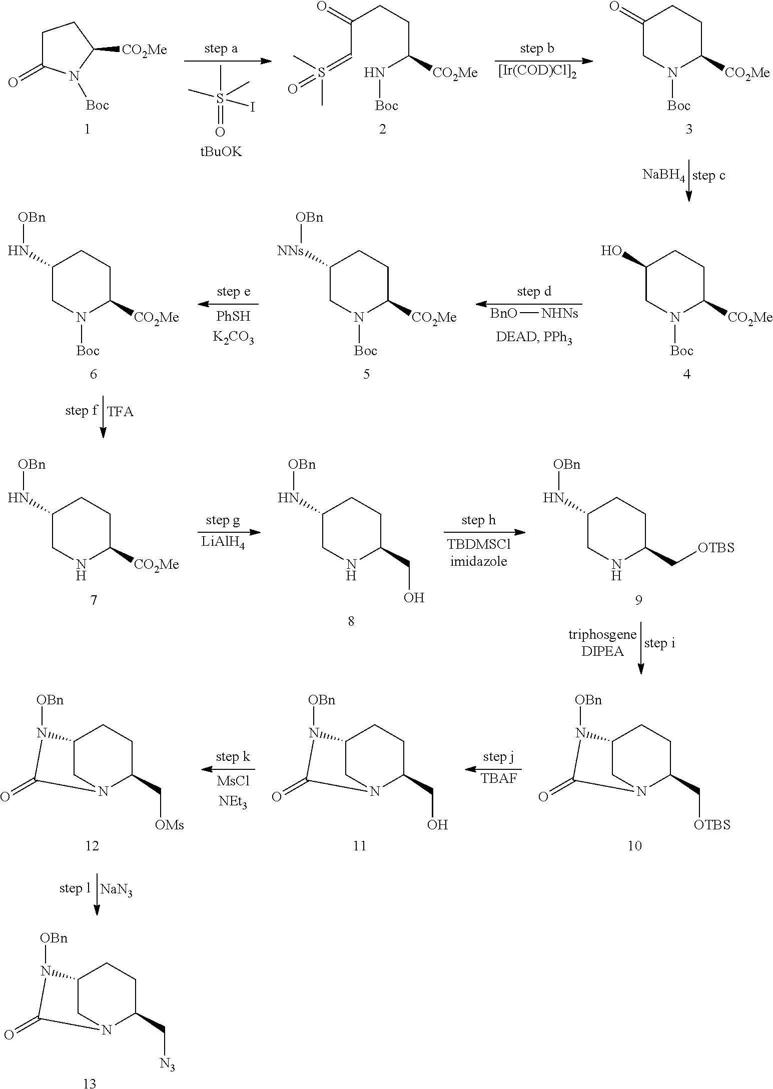

Step a:

A solution of trimethyl sulfoxide iodide (7.70 g, 34.8 mmol) and potassium tert-butoxide (3.46 g, 30.7 mmol) in DMSO (30 mL) was prepared and stirred during 1 h. 1-(tert-butyl) 2-methyl (S)-5-oxopyrrolidine-1,2-dicarboxylate 1 (5 g, 20.5 mmol) was then added and the reaction mixture stirred at room temperature for 3 h. CHCl₃ and water were added and the phases were separated. The organic layer was washed with brine, dried over MgSO4 and concentrated in vacuo to afford 2 as a yellow oil (3.66 g, 53%).

Step b:

[Ir(COD)Cl]₂ (14 mg, 0.02 mmol) was added to a solution of 2 (2.96 g, 8.8 mmol) in DCE (20 mL) and the mixture heated at 80° C. for 48 h in a sealed tube. After cooled down to room temperature, the solution was concentrated under vacuo and the crude product was purified by flash chromatography using cyclohexane/EtOAc (8/2) as eluent to give 3 (1.63 g, 72%) as an orange oil. MS: calculated for C₁₂H₂₀NO₅ [M+H]⁺: 258.1; found: 258.1.

Step c:

NaBH₄ (191 mg, 5.0 mmol) was added at 0° C. to solution of 3 (651 mg, 2.5 mmol) in methanol (10 mL) and the solution was stirred for 2 h at 0° C. The reaction mixture was warm to room temperature and quenched with water. EtOAc was added and the organic layer was washed with brine, dried over MgSO₄ and concentrated in vacuo. The crude product was purified by flash chromatography using cyclohexane/EtOAc (6/4) as eluent to give 4 (596 mg, 91%) as a colorless oil. HRMS: calculated for C₁₂H₂₂NO₅ [M+H]⁺: 260.1498; found: 260.1488.

Step d:

Triphenylphosphine (3 g, 11.6 mmol) and N-nitrosulfonyl-O-benzyl hydroxylamine (2 g, 6.3 mmol) were added to a solution of 4 (1.5 g, 5.8 mmol) in THF (50 mL). DEAD (2.1 mL, 11.6 mmol) was added dropwise and the reaction mixture stirred 24 h at room temperature and concentred in vacuo. The crude product was purified by flash chromatography using cyclohexane/EtOAc (8/2) as eluent to give 5 (2.67 g, 83%) as a colorless oil. HRMS: calculated for C₂₅H₃₂N₃O₉S[M+H]⁺: 550.1859; found: 550.1850.

Step e:

Thiophenol (1.03 mL, 10.0 mmol) and K₂CO₃ (2.8 g, 20.1 mmol) were added to a solution of 5 (3.7 g, 6.7 mmol) in MeCN (80 mL) and the reaction mixture was stirred at room temperature overnight. EtOAc was then added and the organic layer was washed with brine, dried over MgSO₄, and concentrated in vacuo. Purication by flash chromatography using cylclohexane/EtOAc (7/3) as the eluent gave 6 (2.4 g, 98%) as a colorless oil. HRMS: calculated for C₁₉H₂₉N₂O₅ [M+H]⁺: 365.2076; found: 365.2062.

Step f:

Trifluoroacetic acid (5 mL, 60 mmol) was added at 0° C. to a solution of 6 (2.4 g, 6.6 mmol) in DCM (70 mL). The reaction mixture was allowed to warm to room temperature and stirred overnight. The resulting solution was quenched with a saturated solution of NaHCO₃, filtered through a pad of celite and concentrated under vacuo. The crude product was purified by flash chromatography using DCM/MeOH (96/4) as eluent to give 7 (1.7 g, quant.) as a colorless oil. HRMS: calculated for C₁₄H₂₁N₂O₃ [M+H]⁺: 265.1552; found: 265.1552.

Step g:

A solution of 7 (300 mg, 1.1 mmol) in THF (20 mL) was added to a solution of lithium aluminium hydride (2.2 mg, 2.2 mmol, 1 M in THF) in anhydrous THF (20 mL) at 0° C. The solution was stirred for 1 h 30 at 0° C., then quenched with Rochelle's salts. The reaction mixture was filtered through a pad of celite and concentrated under vacuo. The crude residue was purified by chromatography with DCM/MeOH/NH₄OH (8/2/0.5) as the eluent, yielding 8 as a colorless oil (121 mg, 51%). HRMS: calculated for C₁₃H₂₁N₂O₂ [M+H]⁺: 237.1603; found: 237.1599.

Step h:

Imidazole (48 mg, 0.68 mmol) and TBDMSCl (107 mg, 0.68 mmol) were successively added at 0° C. to a solution of 8 (42 mg, 0.17 mmol) in DMF (1 mL). The reaction mixture was stirred at room temperature overnight then evaporated under vacuo. The crude residue was purified by chromatography with cyclohexane/EtOAc (1/9) as the eluent, yielding 9 as a white foam (49 mg, 79%). HRMS: calculated for C₁₉H₃₅N₂O₅Si [M+H]⁺: 351.2467; found: 351.2453.

Step i:

A solution of triphosgene (7 mg, 0.025 mmol) in MeCN (300 μL) was added at 0° C. to a mixture of 9 (17 mg, 0.05 mmol) and DIPEA (42 μL, 0.25 mmol) in MeCN (2 mL). The reaction was stirred 2 h at 0° C. EtOAc was then added and the organic layer was washed with brine, dried over MgSO₄, and concentrated in vacuo. Purication by fash chromatography using cylclohexane/EtOAc (8/2) as the eluent gave 10 (11 mg, 61%) as a colorless oil. HRMS: calculated for C₂₀H₃₃N₂O₃Si [M+H]⁺: 377.5731; found: 377.2260.

Step j:

TBAF (373 μL, 1.36 mmol) was added at 0° C. to a solution of 10 (342 mg, 0.90 mmol) in THF (20 mL). The reaction mixture was stirred 1 h at 0° C., warm to room temperature and concentrated in vacuo. EtOAc was then added and the organic layer was washed with brine, dried over MgSO₄, and concentrated in vacuo. The crude residue was purified by chromatography with cyclohexane/EtOAc (96:4) as the eluent, yielding 11 as a white foam (235 mg, 98%).

Step k:

Methanesulfonyl chloride (5 μL, 0.067 mmol), DMAP (1 mg, 0.0067 mmol) and NEt₃ (20 μL, 0.135 mmol) were added at 0° C. to a solution of 11 (12 mg, 0.045 mmol) in DCM (2 mL). The reaction was stirred 1 h at 0° C. After warmed to room temperature, DCM was added and the organic layer was washed with brine, dried over MgSO₄, and concentrated under reduce pressure to afford 12 (11 mg, 73%).

Step l:

Sodium azide (11 mg, 0.160 mmol) was added to a solution of 12 (11 mg, 0.032 mmol) in DMF (1 mL) and the reaction mixture was stirred overnight at 80° C. EtOAc was then added and the organic layer was washed with brine, dried over MgSO₄, and concentrated in vacuo. Purication by flash chromatography using cylclohexane/EtOAc (7/3) as the eluent gave 13 (7 mg, 78%) as a with foam. HRMS: calculated for C₂₄H₁₈N₅O₂ [M+H]⁺: 288.3250; found: 288.1452.

The compound of formula III.i, wherein X is a sulfur atom and Yₚ is a benzyl group (compound 13′) can be obtained by slightly modifying step i, namely by using thiocarbonyl diimidazole instead of the triphosgene, to afford 10′.

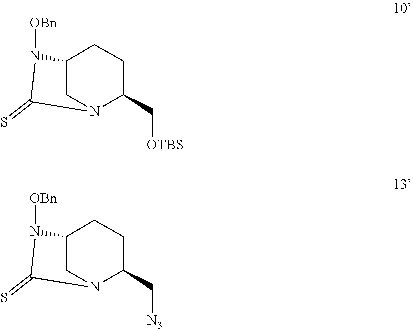 [SMILES: CC(C)(C)[Si](C)(C)OC[C@@H]1CC[C@@H]2CN1C(=S)N2OCc1ccccc1 ; [N-]=[N+]=NC[C@@H]1CC[C@@H]2CN1C(=S)N2OCc1ccccc1]

ii) Stereoisomer (III.ii)

The above-mentioned step c is stereoselective. The compound of formula III.ii, wherein X is an oxygen atom and Yp is a benzyl group (compound 13.ii) can thus be obtained by carrying out the previously detailed successive steps a to I starting from the enantiomer of compound 1, compound (R)-1:

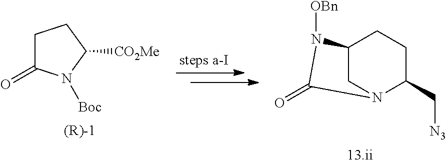 [SMILES: COC(=O)[C@H]1CCC(=O)N1C(=O)OC(C)(C)C ; [N-]=[N+]=NC[C@@H]1CC[C@H]2CN1C(=O)N2OCc1ccccc1]

iii) Stereoisomer (III.iii)

The compound of formula III.iii, wherein X is an oxygen atom and Yₚ is a benzyl group (compound 13.iii) can be obtained by carrying out the multi-steps synthesis detailed for compound 13.ii, in which steps c-e are replaced with an oxyme formation step followed by a reduction step:

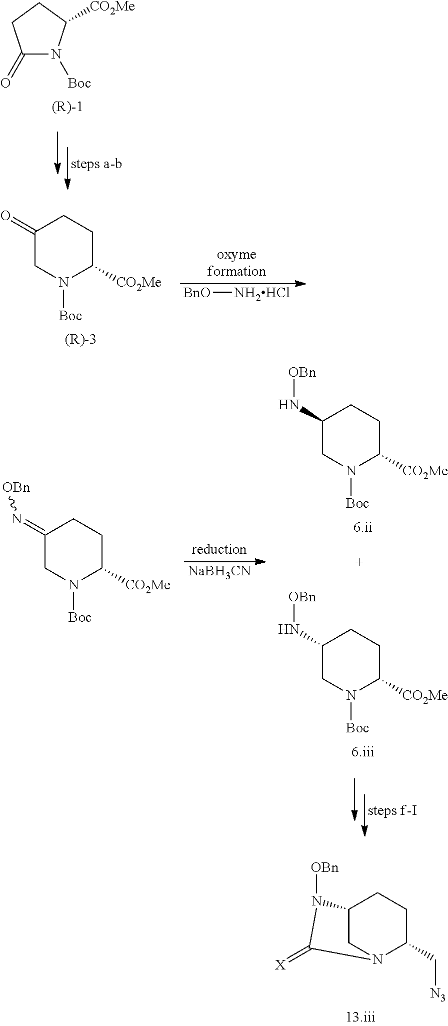

iv) Stereoisomer (III.iv)

The compound of formula III.iv, wherein X is an oxygen atom and Yp is a benzyl group (compound 13.iv) can be obtained by carrying out the multi-steps synthesis detailed for compound 13.i, in which steps c-e are replaced with an oxyme formation step followed by a reduction step:

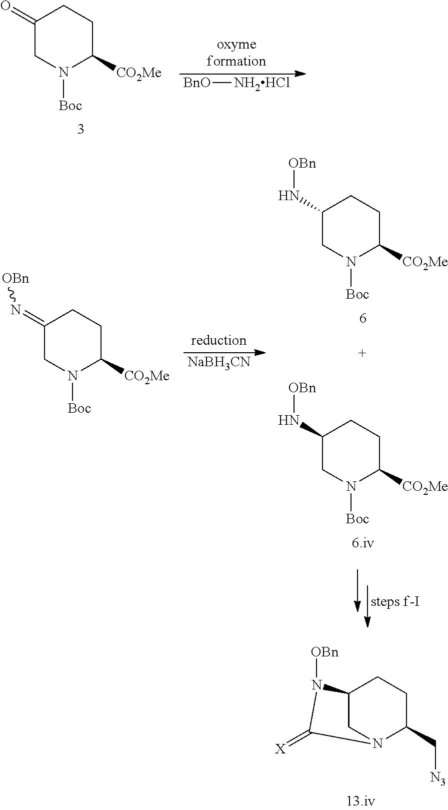

### 1-2. Synthesis of the Compounds of General Formula (I)

i) Compound 1a.i

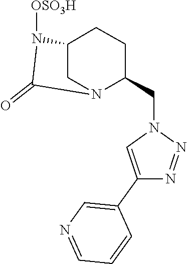

Compound 1a.i was prepared as a sodium salt (compound 1a.i.Na) as follows:

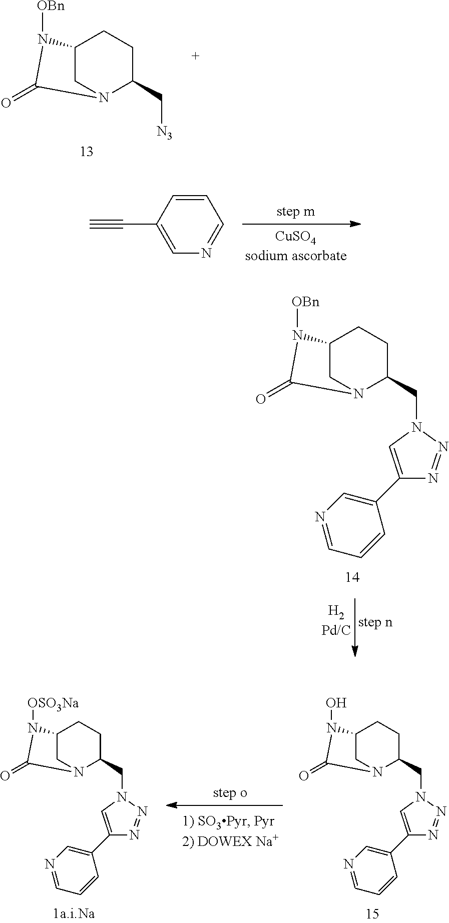

Step m:

To a solution of compound 13 (65 mg, 0.22 mmol) in DMF were successively added 3-ethynylpyridine (47 mg, 0.45 mmol), sodium ascorbate (0.13 mmol, 26 mg, in water (500 μL)) and CuSO₄ (11 mg, 0.06 mmol, in water (500 μL)). The heterogeneous mixture was stirred vigorously overnight at room temperature. EtOAc was then added, the phases were separated, and the aqueous layer was extracted with EtOAc. The combined organic layers were washed with brine, dried over MgSO4, and concentrated under reduced pressure. The crude product was purified by flash chromatography using DCM/MeOH (96/4) as eluent to afford compounds 14 (88 mg, 100%). HRMS: calculated for C₂₁H₂₃N₆O₂ [M+H]⁺: 391.1882; found: 391.1882.

Step n:

10 wt % Pd/C (24 mg, 0.22 mmol) was added to a solution of compound 14 (88 mg, 0.22 mmol) in MeOH (20 mL) and the reaction mixture was stirred 48 h under H₂ atmosphere. Palladium was removed by filtration through Celite® and the filtrate concentrated. Debenzylated compound 15 was used in the next step without further purification. HRMS: calculated for C₁₄H₁₇N₆O₂ [M+H]⁺: 301.1413; found: 301.1412.

Step o:

SO₃ pyrdine complex (300 mg, 1.9 mmol) was added to a solution of 15 (66 mg, 0.22 mmol) in pyridine (2 mL) and the reaction mixture was stirred overnight at room temperature and concentrated under vacuo. The crude was then solubilized in water, filtered on a DOWEX-Na resin and concentrated. The residue was purified by HPLC. The appropriate fractions were collected and lyophilized, to give 1a.i.Na as a white solid (7 mg, 10%, rt=15 min, CH₃CN/H₂O 0:100 to 100:0 over 30 min). HRMS: calculated for C₁₄H₁₅N₆O₅S [M−H]⁺: 379.0825; found: 379.0839. ¹H NMR (500 MHz, D₂O): δ 8.68 (s, 1 H), 8.29 (d, J=5 Hz, 1 H), 8.25 (s, 1 H), 7.99 (d, J=5 Hz, 1 H), 7.33-7.30 (m, 1 H), 4.76-4.71 (m, 1 H), 4.02 (s, 1 H), 3.74 (bs, 1 H), 3.29 (d, J=15 Hz, 1 H), 2.97 (d, J=10 Hz, 1 H), 1.89-1.83 (m, 1 H), 1.82-1.72 (m, 2 H), 1.54-1.48 (m, 1 H), 1.03 (bs, 1 H).

ii) Compound 1a.i11 to compound 1a.i19

Compound 1a.i11 to compound 1a.i19 was prepared as follows:

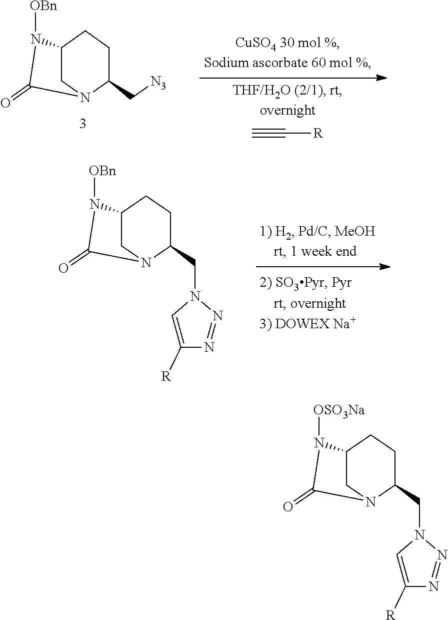

### GENERAL EXPERIMENTAL METHODS

### 1. Synthesis

Reactions were carried out under argon atmosphere and performed using freshly distilled solvents. DCM, DMF and pyridine were dried on calcium hydride. THF was dried on sodium/benzophenone. Unless otherwise specified, materials were purchased from commercial suppliers and used without further purification. Progress of the reactions was monitored by thin-layer chromatography (TLC). TLC was performed using Merck commercial aluminium sheets coated with silica gel 60 F₂₅₄ and detection by charring with phosphomolibdic acid in ethanol followed by heating.

### 2. Purification

Purifications were performed by flash chromatography or preparative high-performance liquid chromatography (HPLC).
Flash chromatography was done on silica gel (60 Å, 180-240 mesh) from Merck.
Preparative HPLC was performed using Shimadzu Prominence system with a

Zorbax Extend-C18 prepHT column (150×21.2 mm, 5 μm) from Agilent. A gradient from 100% of H₂O to 100% of CH₃CN in 30 min was used with a flow rate of 15 mL/min. Products were detected by UV absorption at 214 nm.

### 3. Analysis

Compounds were characterized by NMR, Mass and HPLC.
NMR spectra was recorded on Bruker spectrometers (AM250, Avance II 500 and Avance III HD 4000). Chemical shifts (δ) are reported in parts per million (ppm) and referenced to the residual proton or carbon resonance of the solvents: CDCl₃ (δ 7.26) or D₂O (δ 4.79) for ¹H and CDCl₃ (δ 77.16) for ¹³C. Signals were assigned using 1D (¹H and ¹³C) and 2D (HSQC and COSY) spectra. NMR coupling constants (J) are reported in Hertz (Hz) and splitting patterns are indicated as follows: s (singlet), d (doublet), t (triplet), sx (sextet), dd (doublet of doublet), qd (quartet of doublet), m (multiplet)
Mass spectroscopy (MS) and High-resolution mass spectroscopy (HRMS) was recorded with an ion trap mass analyser under electrospray ionization (ESI) in negative ionization mode detection. MS was performed using Thermo Fisher Scientific LCQ Deca XPMax spectrometer and HRMS was recorded on Thermo Scientific LTQ Orbitrap XL and Bruker MaXis II ETD spectrometers.
HPLC analyses was performed on a Shimadzu Prominence system with an Agilent Zorbax extend C18 column (250×4.6 mm, 5 μm) and UV detection at 214 nm. The injection volume was 20 μL and a gradient from 100% of H₂O+0.1% TFA to 100% of CH₃CN+0.1% TFA in 30 min was used with a flow rate of 1 mUmin.

Compound 1:

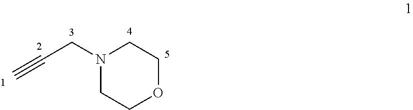

Morpholine (433 μL, 5 mmol) was added at 0° C. to a solution of K₂CO₃ (1.38 g, 10 mmol) in DMF (40 mL). A solution of propargyl bromide 80 wt. % in toluene (517 μL, 6 mmol) was added dropwise and the reaction mixture stirred for 30 min at 0° C. and then at room temperature overnight. EtOAc was then added and the organic layer was washed with 3×H₂O, dried over MgSO₄ and concentrated under vacuum. Purification by flash chromatography using DCM/MeOH (96/4) as the eluant gave the compound 1 as a yellow oil (75 mg, 12%).

Chemical Formula: C₇H₁₁NO

Molecular Weight: 125.17 g.mol⁻¹

¹H NMR (250 MHz, CDCl₃) δ 3.75 (t, J=4.7 Hz, 4H, H₅), 3.29 (d, J=2.4 Hz, 2H, H₃), 2.57 (t, J=4.7 Hz 4H, H₄), 2.27 (t, J=2.4 Hz, 1H, H₁)

¹³C NMR (125 MHz, CDCl₃) δ 78.6 (C₂), 73.5 (C₁), 67.0 (C₅), 52.3 (C₄), 47.3 (C₃)

Compound 2:

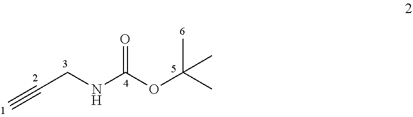

Boc₂O (3.4 g, 15.6 mmol) and DMAP (94 mg, 0.78 mmol) were added at 0° C. to a solution of propargylamine (1 mL, 15.6 mmol) in DCM (60 mL) and the reaction mixture was stirred at room temperature overnight. DCM was then added and the organic layer was washed with brine, dried over MgSO₄ and concentrated under vacuum. Purification by flash chromatography using cyclohexane/EtOAc (95/5) as the eluant gave the compound 2 as a yellow solid (1.33 g, 55%).

Chemical Formula: C₈H₁₃NO₂

Molecular Weight: 155.20 g.mol⁻¹

¹H NMR (500 MHz, CDCl₃) δ 3.87 (s, 2H, H₃), 2.18 (s, 1H, H₁), 1.40 (s, 9H, H₆)

¹³C NMR (125 MHz, CDCl₃) δ 155.4 (C₄), 80.2 (C_{2 or 5}), 80.0 (C_{5 or 2}), 71.3 (C₁), 30.4 (C₃), 28.4 (C₆)

Copper(I)-Catalyzed Azide-Alkyne Cycloaddition Reaction (CuAAC):

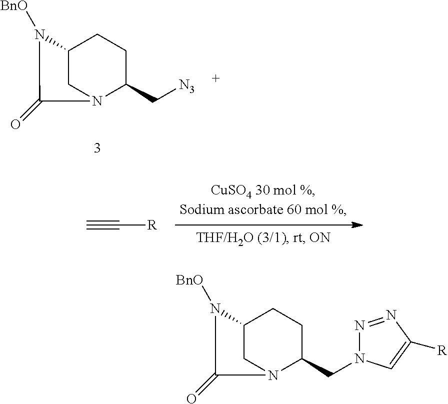

To a solution of 3 in THF, were successively added alkyne (2 eq), sodium ascorbate (0.6 eq, in water) and CuSO₄ (0.3 eq, in water). The heterogeneous mixture was stirred overnight at room temperature. EtOAc was then added, the phases were separated and the aqueous layer was extracted with EtOAc. The combined organic layers were washed with brine, dried over MgSO₄ and concentrated under vacuum. The crude product was purified by flash chromatography to afford the desired product.

Compound 4:

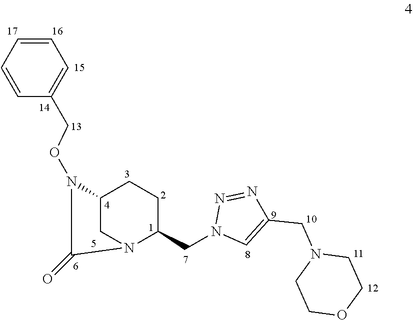 [SMILES: O=C1N2C[C@@H](CC[C@H]2Cn2cc(CN3CCOCC3)nn2)N1OCc1ccccc1]

Following the general procedure for CuAAC, compound 4 was obtained as a yellow oil (74.5 mg, 72%) starting from compound 3 (72 mg, 0.25 mmol) and compound 1 (63 mg, 0.50 mmol).

Chemical Formula: C₂₁H₂₈N₆O₃

Molecular Weight: 412.49 g.mol⁻¹

¹H NMR (500 MHz, CDCl₃) δ 7.61 (s, 1H, ₈), 7.37-7.26 (m, 5H, H_{15,16,17}), 4.96 (d, J=11.5 Hz, 1H, H₁₃), 4.83 (d, J=11.5 Hz, 1H, H13), 4.51-4.42 (m, 2H, H₇), 3.75 (qd, J=7.4, 4.0 Hz, 1 H, H₁), 3.64 (t, J=4.7 Hz, 4H, H₁₂), 3.60 (s, 2H, H10), 3.34-3.32 (m, 1 H, H₄), 2.88 (s, 2H, H₅), 2.45 (t, J=4.7 Hz, 4H, H₁₁), 2.03-1.98 (m, 1H, H₃), 1.91 (sx, J=7.5 Hz, 1H, H₂), 1.66-1.60 (m, 1H, H₃), 1.54-1.48 (m, 1H, H₂)

¹³C NMR (125 MHz, CDCl₃) δ 169.3 (C₆), 144.5 (C₉), 135.7 (C₁₄), 129.2 (C_{15 or 16 or 17}), 128.7 (C_{15 or 16 or 17}), 128.5 (C_{15 or 16 or 17}), 123.0 (C₈), 78.2 (C₁₃), 66.8 (C₁₂), 58.2 (C₄), 56.7 (C₁), 53.6 (C₁₀), 53.4 (C₁₁), 51.7 (C₇), 43.7 (C₅), 20.3 (C₂), 19.6 (C₃) HRMS calculated for C₂₁H₂₉N₆O₃ [M+H]⁺: 413.23011 ; found: 413.22957

Compound 5:

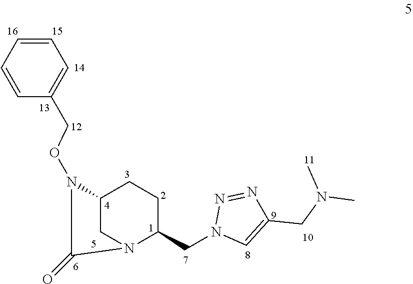 [SMILES: CN(C)Cc1cn(C[C@@H]2CC[C@@H]3CN2C(=O)N3OCc2ccccc2)nn1]

Following the general procedure for CuAAC, compound 5 was obtained as a colorless oil (216 mg, 83%) starting from compound 3 (200 mg, 0.70 mmol) and 3-dimethylamino-1-propyne (151 μL, 1.40 mmol).

Chemical Formula: C₁₉H₂₆N₆O₂

Molecular Weight: 370.46 g.mol⁻¹

¹H NMR (500 MHz, CDCl₃) δ 7.66 (s, 1H, H₈), 7.36-7.27 (m, 5H, H_{14,15,16}), 4.96 (d, J=11.5 Hz, 1H, H₁₂), 4.82 (d, J=11.5 Hz, 1H, H₁₂), 4.52-4.43 (m, 2H, H₇), 3.78-3.73 (m, 1H, H₁), 3.60 (s, 2H, H₁₀), 3.32 (s, 1H, H₄), 2.89 (s, 2H, H₅), 2.25 (s, 6H, H₁₁), 2.02-1.97 (m, 1H, H₃), 1.91 (sx, J=7.5 Hz, 1H, H₂), 1.66-1.59 (m, 1H, H₃), 1.54-1.48 (m, 1H, H₂)

¹³C NMR (125 MHz, CDCl₃) δ 169.4 (C₆), 143.3 (C₉), 135.9 (C₁₃), 129.4 C_{14 or 15 or 16}), 128.9 (C_{14 or 15 or 16}), 128.7 (C_{14 or 15 or 16}), 124.2 (C₈), 78.4 (C₁₂), 58.4 (C₄), 56.9 (C₁), 53.9 (C₁₀), 52.0 (C₇), 44.5 (C₁₁), 43.8 (C₅), 20.4 (C₂), 19.8 (C₃) HRMS calculated for C₁₉H₂₇N₆O₂ [M+H]⁺: 371.21955; found: 371.21900 [α]_{D}: 24.7° (7.5 mg/mL, MeOH)

Compound 6:

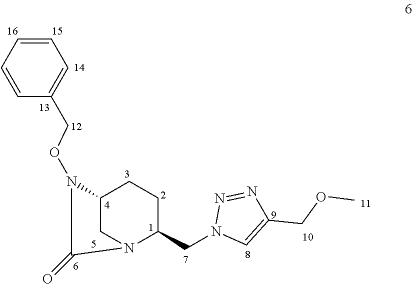 [SMILES: COCc1cn(C[C@@H]2CC[C@@H]3CN2C(=O)N3OCc2ccccc2)nn1]

Following the general procedure for CuAAC, compound 6 was obtained as a colorless oil (215 mg, 86%) starting from compound 3 (200 mg, 0.70 mmol) and methyl propargyl ether (118 μL, 1.40 mmol).

Chemical Formula: C₁₈H₂₃N₅O₃

Molecular Weight: 357.41 g.mol⁻¹

¹H NMR (500 MHz, CDCl₃) δ 7.43-7.33 (m, 5H, H_{14,15,16}), 5.02 (d, J=11.5 Hz, 1H, H₁₂), 4.87 (d, J=11.5 Hz, 1H, H₁₂), 4.57-4.50 (m, 4H, H_{7 and} 10), 3.84-3.82 (m, 1H, H₁), 3.43 (s, 3H, H₁₁), 3.35 (q, J=3.0 Hz, 1H, H₄), 2.90 (dd, J=17.3, 11.9 Hz, 2H, H₅ ), 2.09-2.04 (m, 1 H, H₃), 1.97 (sx, J=7.4 Hz, 1 H, H₂), 1.70-1.63 (m, 1H, H₃), 1.60-1.54 (m, 1H, H₂) *H₈ not visible on the ¹H NMR spectrum

¹³C NMR (125 MHz, CDCl₃) δ 169.3 (C₆), 135.9 (C₁₃), 129.4 (C_{14 or 15 or 16}), 128.9 (C_{14 or 15 or 16}), 128.7 (C_{14 or 15 or 16}), 78.4 (C₁₂), 66.0 (C₁₀), 58.5 (C₁₁), 58.4 (C₄), 56.7 (C₁), 52.3 (C₇), 43.8 (Cs), 20.4 (C₂), 19.8 (C₃) C₈ and C₉ not visible on the ¹³C NMR spectrum HRMS calculated for C₁₈H₂₄N₅O₃ [M+H]⁺: 358.18791; found: 358.218771 [α]_{D}: 27.7° (6.6 mg/mL, MeOH)

Compound 7:

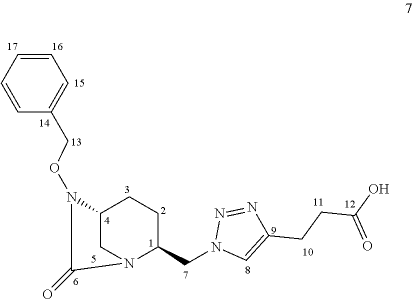 [SMILES: O=C(O)CCc1cn(C[C@@H]2CC[C@@H]3CN2C(=O)N3OCc2ccccc2)nn1]

Following the general procedure for CuAAC, compound 7 was obtained as a colorless oil (202 mg, 75%) starting from compound 3 (200 mg, 0.70 mmol) and 4-pentynoic acid (137 mg, 1.40 mmol).

Chemical Formula: C₁₉H₂₃N₅O₄

Molecular Weight: 385.42 g.mol⁻¹

¹H NMR (500 MHz, CDCl₃) 6 7.41-7.34 (m, 5H, H15, 16, 17), 5.01 (d, J=11.4 Hz, 1H, H₁₃), 4.87 (d, J=11.4 Hz, 1H, H₁₃), 4.56-4.50 (m, 2H, H₇), 3.83-3.82 (m, 1H, H₁), 3.36 (s, 1H, H₄), 3.06 (s, 2H, H₁₀), 2.94 (s, 2H, H₅), 2.79 (s, 2H, H₁₁), 2.09-2.00 (m, 1H, H₃), 2.00-1.89 (m, 1H, H₂), 1.73-1.62 (m, 1H, H₃), 1.62-1.51 (m, 1H, H₂) *Hₐ not visible on the ¹H NMR spectrum

¹³C NMR (125 MHz, CDCl₃) δ 175.8 (C₁₂), 169.4 (C₆), 135.8 (C₁₄), 129.4 (C_{15 or 16 or 17}), 128.9 (C_{15 or 16 or 17}), 128.7 (C_{15 or 16 or 17}), 78.4 (C₁₃), 58.5 (C₄), 56.6 (C₁), 52.3 (C₇), 43.9 (C₅), 33.3 (C₁₁), 20.9 (C₁₀), 20.4 (C₂), 19.8 (C₃) *C₈ and C₉ not visible on the ¹³C NMR spectrum HRMS calculated for C₁₉H₂₄N₅O₄ [M+H]⁺: 386.18283; found: 386.18228 [α]_{D}: 23.8° (7.9 mg/mL, MeOH)

Compound 8:

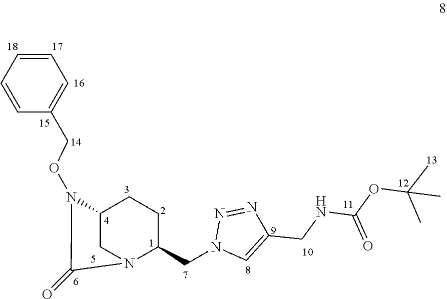 [SMILES: CC(C)(C)OC(=O)NCc1cn(C[C@@H]2CC[C@@H]3CN2C(=O)N3OCc2ccccc2)nn1]

Following the general procedure for CuAAC, compound 8 was obtained as a colorless oil (289 mg, 93%) starting from compound 3 (200 mg, 0.70 mmol) and compound 2 (217 mg, 1.40 mmol).

Chemical Formula: C₂₂H₃₀N₆O₄

Molecular Weight: 442.52 g.mol⁻¹

¹H NMR (500 MHz, CDCl₃) δ 7.62 (s, 1H, H₈), 7.39-7.30 (m, 5H, H_{16, 17, 18}), 4.98 (d, J =11.5 Hz, 1H, H₁₄), 4.84 (d, J=11.5 Hz, 1H, H₁₄), 4.53-4.42 (m, 2H, H₇), 4.34 (d, J=5.9 Hz, 2H, H₁₀), 3.79-3.74 (m, 1H, H₁), 3.34-3.32 (m, 1 H, H₄), 2.89 (s, 2H, H₅), 2.04-1.99 (m, 1H, H₃), 1.93 (sx, J=7.5 Hz, 1 H, H2), 1.67-1.60 (m, 1 H, H₃), 1.54-1.48 (m, 1H, H₂), 1.41 (s, 9H, H₁₃)

¹³C NMR (125 MHz, CDCl₃) δ 169.3 (C₆), 155.9 (C₁₁), 145.9 (C₉), 135.8 (C₁₅), 129.3 (C_{16 or 17 or 18}), 128.8 (C_{16 or 17 or 18}), 128.6 (C_{16 or 17 or 18}), 122.3 (C₈), 79.7 (C₁₂), 78.2 (C₁₄), 58.3 (C₄), 56.7 (C₁), 51.7 (C₇), 43.8 (C₅), 36.3 (C₁₀), 28.4 (C₁₃), 20.3 (C₂), 19.7 (C₃) HRMS calculated for C₂₂H₃₁ N₆O₄ [M+H]⁺: 443.24068; found: 443.23941 [α]_{D}: 20.5° (5.4 mg/mL, MeOH)

Compound 9:

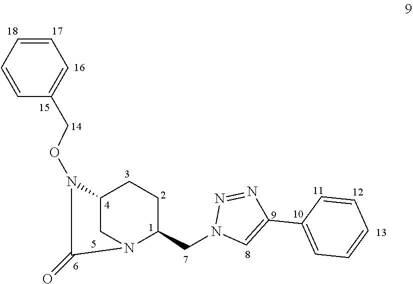 [SMILES: O=C1N2C[C@@H](CC[C@H]2Cn2cc(-c3ccccc3)nn2)N1OCc1ccccc1]

Following the general procedure for CuAAC, compound 9 was obtained as a colorless oil (232 mg, 85%) starting from compound 3 (200 mg, 0.70 mmol) and phenylacetylene (154 82 L, 1.40 mmol).

Chemical Formula: C₂₂H₂₃N₅O₂

Molecular Weight: 389.46 g.mol⁻¹

¹H NMR (500 MHz, CDCl₃) δ 7.97 (s, 1H, H₈), 7.84-7.82 (m, 2H, H₁₁), 7.43-7.32 (m, 8H, H_{12, 13, 16, 17, 18}), 5.03 (d, J=11.5 Hz, 1H, H₁₄), 4.88 (d, J=11.5 Hz, 1 H, H₁₄), 4.63-4.54 (m, 2H, H₇), 3.91-3.86 (m, 1H, H₁), 3.35 (q, J=2.9 Hz, 1H, H₄), 2.93 (q, J=11.9 Hz, 2H, H₅), 2.10-2.06 (m, 1H, H₃), 2.03-1.96 (m, 1H, H₂), 1.72-1.68 (m, 1H, H₃), 1.66-1.60 (m, 1H, H₂)

¹³C NMR (125 MHz, CDCl₃) δ 169.3 (C₆), 148.2 (C₉), 135.9 (C₁₅), 130.3 (C₁₀), 129.4 (C_{12 or 13 or 16 or 17 or 18}), 129.0 (C_{12 or 13 or 16 or 17 or 18}), 128.9 (C_{12 or 13 or 16 or 17 or 18}), 128.7 (C_{12 or 13 or 16 or 17 or 18}), 128.5 (C_{12 or 13 or 16 or 17 or 18}), 126.0 (C₁₁), 120.3 (C₈), 78.4 (C₁₄), 58.4 (C₄), 56.7 (C₁), 52.2 (C₇), 43.9 (C₅), 20.4 (C₂), 19.8 (C₃) HRMS calculated for C₂₂H₂₄N₅O₂ [M+H]⁺: 390.19300; found: 390.19165

Compound 10:

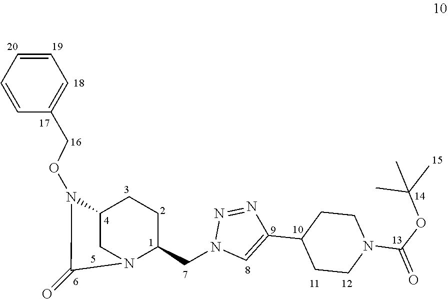 [SMILES: CC(C)(C)OC(=O)N1CCC(c2cn(C[C@@H]3CC[C@@H]4CN3C(=O)N4OCc3ccccc3)nn2)CC1]

Following the general procedure for CuAAC, compound 10 was obtained as a colorless oil (224 mg, 64%) starting from compound 3 (200 mg, 0.70 mmol) and 1-boc-4-ethynylpiperidine (293 mg, 1.40 mmol).

Chemical Formula: C₂₆H₃₆N₆O₄

Molecular Weight: 496.61 g.mol⁻¹

¹H NMR (500 MHz, CDCl₃) δ 7.42-7.33 (m, 5H, C_{18, 19, 20}), 5.02 (d, J=11.5 Hz, 1H, H₁₆), 4.87 (d, J=11.4 Hz, 1H, H₁₆), 4.55 (s, 2H, H₇), 4.17 (d, J=12.0 Hz, 2H, H₁₂), 3.84 (s, 1H, H₁), 3.36 (s, 1H, H₄), 2.93 (m, 5H, H_{5, 10, 12}), 2.17-1.97 (m, 4H, H_{2, 3, 11}), 1.73-1.59 (m, 4H, H_{2, 3, 11}), 1.47 (s, 9H, H₁₅) H₈ not visible on the ¹H NMR spectrum

¹³C NMR (125 MHz, CDCl₃) δ 169.2 (C₆), 154.7 (C₁₃), 135.7 (C₁₇), 129.1 (C_{18 or 19 or 20}), 128.7 (C_{18 or 19 or 20}), 128.5 (C_{18 or 19 or 20}), 79.4 (C₁₄), 78.1 (C₁₆), 58.3 (C₄), 56.5 (C₁), 52.2 (C₇), 43.6 (C₅ and C12), 33.6 (C₁₀), 31.4 (C₁₁), 28.4 (C₁₅), 20.4 (C₂), 19.6 (C₃) *C₈ and C₉ not visible on the ¹³C NMR spectrum

Introduction of Sodium Sulphite: General Procedure

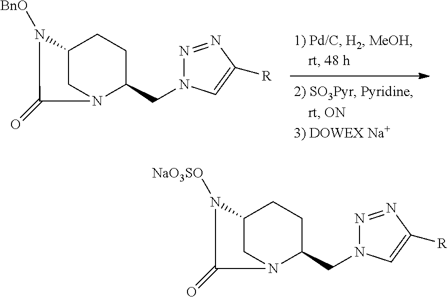

Protected DBO
1. 10 wt. % Pd/C (1 eq) was added to a solution of protected DBO in MeOH and the reaction mixture was stirred under H₂ for 48 h at room temperature. Palladium was removed by filtration through celite and the filtrate concentrate.
2. SO₃-pyridine complex (6 eq) was added to a solution of deprotected compound in pyridine and the reaction mixture was stirred 2 h at room temperature. Additional SO₃Pyr (2 eq) was then added, stirred overnight at room temperature and pyridine was removed under reduced pressure.
3. The crude product was solubilized in water, filtered, eluted on Dowex-Na resin io with H₂O and lyophilized. The residue was dissolved in EtOH, filtered and the filtrate was concentrated under vacuum. HPLC purification gave the desired product.

Compound 1a.i11:

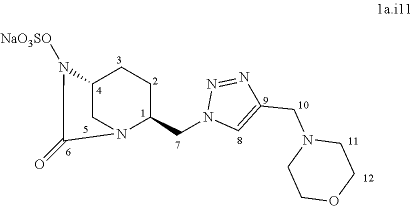

Following the general procedure for the introduction of sodium sulphite, compound 1a.i11 was obtained as a yellow foam (6 mg, 8%) starting from compound 4 (74 mg, 0.18 mmol).

Chemical Formula: C₁₄H₂₁N₆NaO₆S

Molecular Weight: 424.41 g.mol⁻¹

¹H NMR (250 MHz, D₂O) δ 8.08 (s, 1H, H₈), 4.87 (m, 2H, H₇), 4.20-4.18 (m, 1H, H₄), 3.90-3.84 (m, 3H, H_{1,10}), 3.75-3.72 (m, 4H, H₁₂), 3.46 (d, J=12.3 Hz, 1H, H₅), 3.15-3.08 (m, 1H, H₅), 2.79-2.72 (m, 4H, H₁₁), 2.08-1.85 (m, 3H, H_{2,3}), 1.72-1.63 (m, 1H, H₂) HRMS calculated for C₄H₂₁N₆O₆S [M−H]: 401. 12433; found: 401.12483

Compound 1a.i12:

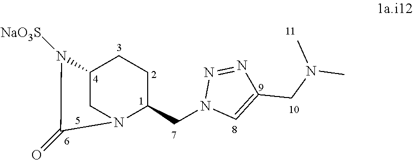

Following the general procedure for the introduction of sodium sulphite, compound 1a.i12 was obtained as a white powder (4.5 mg, 2%) starting from compound 5 (216 mg, 0.58 mmol).

Chemical Formula: C₁₂H₉N₆NaO₅S

Molecular Weight: 382.37 g.mol⁻¹

¹H NMR (500 MHz, D₂O) δ 8.37 (s, 0.6H, H₈), 8.25 (s, 0.4H, H₈), 4.90-4.83 (m, 1H, H₇), 4.63-4.58 (m, 1H, H₇), 4.17 (s, 1H, H₄), 3.84-3.81 (m, 1H, H₁), 3.46-3.42 (m, 1H, H₅), 3.11 (d, J=12.5 Hz, 1H, H₅), 3.06 (s, 6H, H₁₁), 2.81 (s, 2H, H₁₀), 1.98-1.87 (m, 3H, H_{2,3}), 1.68-1.66 (m, 1H, H₂) MS calculated for C₁₂H₁₉N₆O₅[M−H]: 359.11; found: 359.33

Compound 1a.i13:

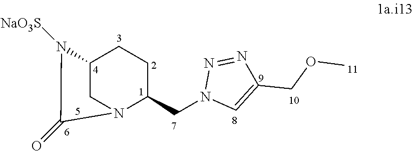

Following the general procedure for the introduction of sodium sulphite, compound 1a.i13 was obtained as a colorless foam (28 mg, 13%) starting from compound 6 (210 mg, 0.59 mmol).

Chemical Formula: C₁₁H₁₆N₅NaO₆S

Molecular Weight: 369.33 g.molhu −1

¹H NMR (500 MHz, D₂O) δ 8.17 (s, 1H, Hs), 4.96 (dd, J=14.8, 10.3 Hz, 1H, H₇), 4.73 (dd, J=14.8, 5.7 Hz, 1H, H₇), 4.68 (s, 2H, H₁₀), 4.32-4.30 (m, 1H, H₄), 4.00-3.95 (m, 1H, H₁), 3.54 (d, J=12.3 Hz, 1H, H₅), 3.45 (s, 3H, H₁₁), 3.26-3.23 (m, 1H, H₅), 2.19-2.12 (m, 1H, H₃), 2.08-1.98 (m, 2H, H_{2,3}), 1.80-1.73 (m, 1H, H₂)

¹³C NMR (125 MHz, D₂O) δ 170.1 (C₆), 144.0 (C₉), 125.4 (C₉), 64.4 (C₁₀), 60.1 (C₄), 57.9 (C₁), 57.5 (C₁₁), 50.7 (C₇), 43.6 (C₅), 19.7 (C₂), 18.8 (C₃) HRMS calculated for C₁₁H₁₆N₅O₆S [M−H]: 346.08213; found: 346.08185 Rt 13.3 min

Compound 1a.i14:

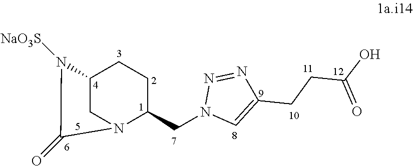

Following the general procedure for the introduction of sodium sulphite, compound 1a.i14 was obtained as a colorless foam (34 mg, 17%) starting from compound 7 (194 mg, 0.50 mmol).

Chemical Formula: C₁₂H₁₆N₅NaO₇S

Molecular Weight: 397.34 g.mol⁻¹

¹H NMR (500 MHz, D₂O) δ 7.88 (s, 1H, H₈), 4.75-4.65 (m, 2H, H₇), 4.30-4.28 (m, 1H, H₄), 3.98-3.95 (m, 1H, H₁), 3.52 (d, J=12.3 Hz, 1H, H₅), 3.25-3.21 (m, 1H, H₅), 3.00 (t, J=7.6 Hz, 2H, H₁₀), 2.58 (t, J=7.6 Hz, 2H, H₁₁), 2.15-2.11 (m, 1H, H₃), 2.05-1.96 (m, 2H, H_{2,3}), 1.77-1.71 (m, 1H, H₂)

¹³C NMR (125 MHz, D₂O) δ 181.7 (C₁₂), 170.1 (C₆), 148.0 (C₉), 123.3 (C₈), 60.0 (C₄), 57.8 (C₁), 50.5 (C₇), 43.7 (C₅), 36.8 (C₁₁), 21.8 (C₁₀), 19.6 (C₂), 18.8 (₃) HRMS calculated for C₁₂H₁₆N₅O₇S [M−H]: 374.07704; found: 374.07651 Rt 13.3 min

Compound 1a.i15:

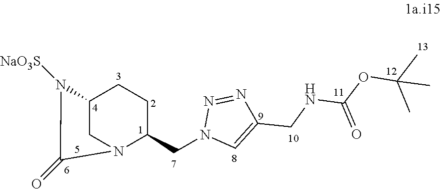

Following the general procedure for the introduction of sodium sulphite, compound 1a.i15 was obtained as a white solid (85 mg, 29%) starting from compound 8 (283 mg, 0.64 mmol).

Chemical Formula: C₁₅H₂₃N₆NaO₇S

Molecular Weight: 454.43 g.mol⁻¹

¹H NMR (500 MHz, D₂O) δ 8.01 (s, 1H, H₈), 4.92 (dd, J=14.8, 10.3 Hz, 1H, H₇), 4.69 (dd, J=14.8, 5.7 Hz, 1H, H₇), 4.39 (s, 2H, H₁₀), 4.31-4.29 (m, 1H, H₄), 3.95 (m, 1H, H₁), 3.53 (d, J=12.4 Hz, 1H, H₅), 3.23 (m, 1H, H₅), 2.16-2.11 (m, 1H, H₃), 2.06-1.97 (m, 2H, H_{2,3}), 1.79-1.74 (m, 1H, H₂), 1.47 (s, 9H, H₁₃)

¹³C NMR (125 MHz, D₂O) δ 170.1 (C₆), 158.0 (C₁₁), 146.0 (C₉), 123.8 (C₈), 81.5 (C₁₂), 60.1 (C₄), 57.9 (C₁), 50.7 (C₇), 43.6 (C₅), 35.4 (C₁₀), 27.6 (C₁₃), 19.7 (C₂), 18.8 (C₃) HRMS calculated for C₁₅H₂₃N₆O₇S [M−H]: 431.13489; found: 431.13669 Rt 18.1 min

Compound 1a.i16:

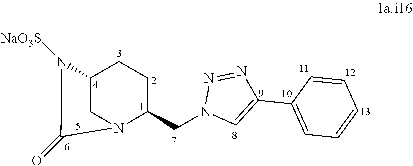

Following the general procedure for the introduction of sodium sulphite, compound 1a.i16 was obtained as a white powder (44.5 mg, 19%) starting from compound 9 (226 mg, 0.58 mmol).

Chemical Formula: C₁₅H₁₆N₅NaO₅S

Molecular Weight: 401.37 g.mol⁻¹

¹H NMR (500 MHz, D₂O) δ 8.28 (s, 1H, H₈), 7.78-7.76 (m, 2H, H₁₁), 7.54-7.51 (m, 2H, H₁₂), 7.48-7.44 (m, 1H, H₁₃), 4.88-4.83 (m, 1H, H₇), 4.62 (dd, J=14.7, 5.7 Hz, 1H, H₇), 4.29 (d, J=3.1 Hz, 1H, H₄), 3.95-3.91 (m, 1H, H₁), 3.50 (d, J=12.3 Hz, 1H, H₅), 3.22 (d, J=12.7 Hz, 1H, H₅), 2.13-2.09 (m, 1H, H₃), 2.04-1.95 (m, 2H, H_{2,3}), 1.75-1.69 (m, 1H, H₂)

¹³C NMR (125 MHz, D₂O) δ 170.1 (C₆), 147.6 (C₉), 129.4 (C₁₀), 129.2 (C₁₂), 128.8 (C₁₃), 125.6 (C₁₁), 122.3 (C₈), 60.1 (C₄), 57.8 (C₁), 50.8 (C₇), 43.6 (C₅), 19.7 (C₂), 18.8 (C₃) MS calculated for C₁₅H₁₆N₅O₅S [M−H]: 378.09; found: 378.33 Rt 19.4 min

Compound 1a.i17:

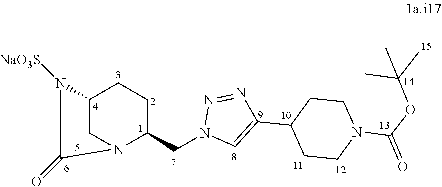

Following the general procedure for the introduction of sodium sulphite, compound 1a.i17 was obtained as a white powder (86 mg, 39%) starting from compound 10 (218 mg, 0.44 mmol).

Chemical Formula: C₁₃H₂₃N₆NaO₇S

Molecular Weight: 508.53 g.mol⁻¹

1H NMR (500 MHz, D₂O) δ7.94 (s, 1H, H₈), 4.90 (dd, J=14.7, 10.2 Hz, 1H, H₇), 4.67 (dd, J=14.7, 5.8 Hz, 1H, H₇), 4.31-4.29 (m, 1H, H₄), 4.13 (d, J=12.7 Hz, 2H, H₁₂), 3.97-3.93 (m, 1H, H₁), 3.51 (d, J=12.3 Hz, 1H, H₅), 3.22 (d, J=12.3 Hz, 1 H, H₅), 3.08-3.01 (m, 3H, H_{10,12}), 2.17-2.11 (m, 1H, H₃), 2.07-1.97 (m, 4H, H2, 3,11), 1.77-1.71 (m, 1H, H₂), 1.66-1.59 (m, 2H, H₁₁), 1.51 (s, 9H, H₁₅)

^{—}C NMR (125 MHz, D₂O) δ 170.1 (C₆), 156.6 (C₁₃), 152.0 (C₉), 122.4 (C₈), 81.7 (C₁₄), 60.1 (C₄), 57.9 (C₁), 50.6 (C₇), 43.7 (C₅ and C₁₂), 32.5 (C₁₀), 31.0 (C11), 27.7 (C₁₅), 19.7 (C₂), 18.8 (C₃) MS calculated for C₁₃H₂₉N₆O₇S [M−H]: 485.18; found: 485.40

Compound 1a.i18:

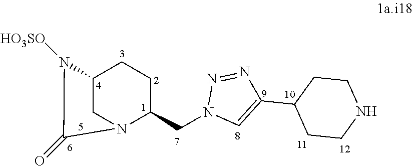

TFA (24 μL, 0.29 mmol) was added dropwise at 0° C. to a solution of 17 (12 mg, 0.02 mmol) in DCM (240 μL). The reaction mixture was stirred for 2 h at 0° C. and concentrated under vacuum. HPLC purification gave the compound 18 as a white solid (1 mg, 6%).

Chemical Formula: C₁₄H₂₂N₆O₅S

Molecular Weight: 386.43 g.mol⁻¹ HRMS calculated for C₁₄H₂₁N₆O₅S [M−H]⁺: 385.12941; found: 385.13052

Compound 19:

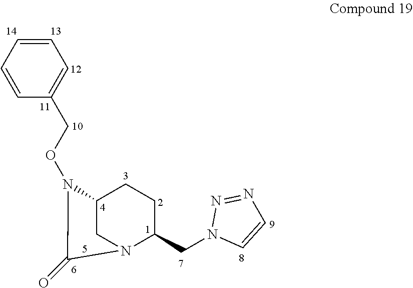 [SMILES: O=C1N2C[C@@H](CC[C@H]2Cn2ccnn2)N1OCc1ccccc1]

1H-1,2,3-triazole (133 μL, 2.29 mmol) was added to a solution of tBuOK (257 mg, 2.29 mmol) in acetonitrile (24 ml). A solution of ((2S,5R)-6-(benzyloxy)-7-oxo-1,6-diazabicyclo[3.2.1]octan-2-yl)methyl methanesulfonate (388 mg, 1.14 mmol) in acetonitrile (18 ml) was then added dropwise and the reaction mixture was stirred for 15 h at 90° C. DCM was then added and the organic layer was washed with H₂O and brine, dried over MgSO₄ and concentrated under vacuum. Purification by flash chromatography using cyclohexane/EtOAc (9/1) as the eluant gave the compounds 19 (136 mg, 37%) as orange solids.

Chemical Formula: C₁₆H₁₉N₅O₂

Molecular Weight: 313.36 g.mol⁻¹

¹NMR (19) (500 MHz, CDCl₃) δ 7.72 (d, J=0.8 Hz, 1H, H₉), 7.66 (d, J=0.8 Hz, 1H, H₈), 7.39-7.30 (m, 5H, H_{12, 13, 14}), 4.98 (d, J=11.5 Hz, 1H, H₁₀), 4.84 (d, J=11.5 Hz, 1H, H₁₀), 4.53 (qd, J=14.2, 7.5 Hz, 2H, H₇), 3.81-3.76 (m, 1H, H₁), 3.36-3.34 (m, 1H, H₄), 2.90 (s, 2H, H₅), 2.05-1.99 (m, 1H, H₃), 1.93 (dq, J=15.1, 7.5 Hz, 1H, H₂), 1.69-1.62 (m, 1H, H₃), 1.57-1.51 (m, 1H, H₂).

Compound 1a.i19:

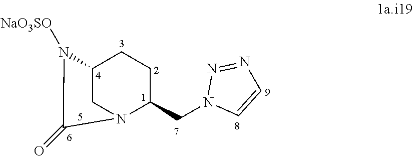

Following the general procedure for the introduction of sodium sulphite, compound 1a.i19 was obtained as a white foam (14 mg, 8%) starting from compound 19 (132 mg, 0.42 mmol).

Chemical Formula: C₉H₁₂N₅NaO₅S

Molecular Weight: 325.27 g.mol⁻¹ HRMS calculated for C₉H₁₂N₅O₅S [M−H]⁻302.05591; found: 302.05670

iii) Compound 2a.i

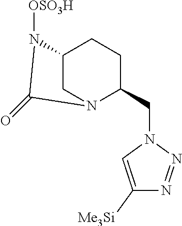

Compound 2a.i was prepared as a sodium salt by carrying out the previously detailed successive steps m to o starting from ethynyltrimethylsilane. HRMS: calculated for C₁₂H₂₀N₅O₅SSi [M−H]⁺: 374.0954; found: 374.0942.

¹H NMR (500 MHz, D₂O): δ8.07 (s, 1 H), 4.20 (bs, 1 H), 3.89-3.86 (m, 1 H), 3.43-3.40 (m, 1 H), 3.15 (bs, 2 H), 2.08-2.01 (m, 1 H), 1.95-1.90 (m, 1 H), 1.69-1.61 (m, 1 H), 1.23 (bs, 2 H), 0.26 (s, 9 H).

### II. β-lactamase Inhibitory Activity of the Compounds According to the Invention

### II-1. Material and Methods

The minimal inhibitory concentrations (MICs) of amoxicillin in the presence or absence of DBOs (15 μM) were determined by the microdilution method in 96-well plates, as described in Dubee et al., Antimicrob. Agents Chemother. 2015, 59, 2938-2941, on line supplement data. Briefly, approximately 5×10⁵ colony-forming units (CFU) per mililiter were inoculated into Middlebrook 7H9 broth supplemented with 10% (vol/vol) oleic acid, albumin, dextrose, catalase (OADC; BD-Difco) and 0.05% (vol/vol) Tween 80 (Sigma) (7H9sB) containing two-fold dilutions of β-lactams in the 0.5 to 256 μg/ml range. Microplates were incubated at 30° C. for 72 h and the MIC was defined as the lowest antibiotic concentration that prevented visible bacterial growth.

MIC determination. DBOs were used at equimolar concentrations (15 μM), corresponding to 4 μg/ml for avibactam.

For M. abscessus CIP104536 and its Δbla_{Mab} derivative, the MICs of amoxicillin were determined in the presence or absence of DBOs by the microdilution method in 96-well round-bottom microplates. The growth medium was a Middlebrook 7H9 broth supplemented with 10% (vol/vol) of OADC supplement, which contains oleic acid, albumin, dextrose, catalase, and 0.05% (vol/vol) Tween 80. This growth medium, containing two-fold dilutions of amoxicillin in the 0.5 to 256 μg/ml range, was inoculated with 1×10⁵ colony-forming units (CFUs) (final volume 200 μl). Microplates were incubated at 30° C. for 48 h and the MIC was defined as the lowest antibiotic concentration that prevented visible bacterial growth.

For M. tuberculosis, the antibacterial activity of amoxicillin in the presence or absence of DBOs was determined by the microdilution method. Briefly, M. tuberculosis H37Rv and its ΔblaC derivative were grown to exponential phase at 37° C. in Middlebrook 7H9 broth containing 0.2% glycerol and 10% of OADC supplement (vol/vol). This growth medium, containing two-fold dilutions of amoxicillin in the 0.125 to 512 μg/ml range, was inoculated with 1×10⁵ CFUs (final volume 200 μ). After 9 days of incubation at 37° C., resazurin (0.0025%, wt/vol) was added to each well and the plates were further incubated overnight. The MIC was defined as the lowest drug concentration that prevented the resazurin color change from blue to pink.

Protein purification for kinetics analysis. The β-lactamases (Bla_{Mab} and BlaC) and the L,D-transpeptidases (Ldtₜₘ and Ldt_{Mt2}) were produced in E. coli BL21(DE3) harboring plasmids pET-TEVΩbla_{Mab} (Soroka, D., et al., Characterization of broad-spectrum Mycobacterium abscessus class A beta-lactamase. J Antimicrob Chemother, 2014), pET-TEVΩblaC (Soroka, D., et al., Characterization of broad-spectrum Mycobacterium abscessus class A beta-lactamase. J Antimicrob Chemother, 2014), pET-TEVΩ/dt_{fm} (Triboulet, S., et al., Kinetic features of L,D-transpeptidase inactivation critical for beta-lactam antibacterial activity. PLoS One, 2013), and pET-TEVΩ/dt_{Mt2} (Cordillot, M., et al., In vitro cross-linking of Mycobacterium tuberculosis peptidoglycan by L,D-transpeptidases and inactivation of these enzymes by carbapenems. Antimicrob Agents Chemother, 2013). Bacteria were grown in brain-heart infusion broth containing kanamycin (50 mg/L). Soluble forms of BlaMab (residues 31-289), BlaC (39-306), Ldtₜₘ (341-466) and Ldt_{Mt2} (55-408) were purified from clarified lysates by metal affinity and size-exclusion chromatography in 25 mM Tris-HCl (pH 7.5) containing NaCl 300 mM (for Bla_{Mab} and BlaC) or in 100 mM sodium phosphate (pH 6.4) containing NaCl 300 mM (for Ldtₜₘ and Ldt_{Mt2}). The purified enzymes were concentrated by ultrafiltration (Amicon Ultra-4 centrifugal filter devices, Millipore) and stored at −65° C. in the same buffers.

Determination of kinetic parameters. Kinetic parameters for the carbamoylation of β-lactamases by DBOs (k₂/K, and k₂) were determined by spectrophotometry at 20° C. using nitrocefin (100 μM) in 2-(N-morpholino)ethanesulfonic acid (MES; 100 mM; pH 6.4), as previously described in Dubee, V., et al., beta-Lactamase inhibition by avibactam in Mycobacterium abscessus. Journal of Antimicrobial Chemotherapy, 2015. Kinetics constants were deduced from a minimum of six progress curves with various concentrations of DBOs, which were obtained in a minimum of two independent experiments. For inactive or weakly active compounds, the highest DBO concentration tested was 100 μM.

Inhibition of L,D-transpeptidases Ldtₜₘ and Ldt_{Mt2} by DBOs. L,D-transpeptidase inhibition was monitored using the hydrolysis of nitrocefin (Edoo, Z., M. Arthur, and J. E. Hugonnet, Reversible inactivation of a peptidoglycan transpeptidase by a beta-lactam antibiotic mediated by beta-lactam-ring recyclization in the enzyme active site. Sci Rep, 2017). Ldtₜₘ or Ldt_{Mt2} (10 μM) was incubated with DBOs (0, 10, 25, 50, 100, or 200 μM) in 100 mM sodium phosphate (pH 6.0) for 260 min at 37° C. before addition of nitrocefin (50 μM). The initial rate of nitrocefin hydrolysis was determined at 20° C. (Δ_{ϵ486 nm}=15,200 M⁻¹ cm⁻¹). Inhibition was evaluated by determining the residual (%) rate of initial hydrolysis of nitrocefin with 100% corresponding to L,D-transpeptidases incubated in the absence of inhibitor in the same conditions.

### II-2. Results

The results obtained are presented in Table 1.

TABLE 1
Evaluation of DBOs (15 μM) as β-lactamase inhibitors in mycobacteria.
The concentration of 15 μM corresponds to 6 μg/ml of 1a.i.Na.

| | MIC of amoxicillin (μg/ml) | | | |
|---|---|---|---|---|
| | M. tuberculosis | | M. abscessus | |
| | | H37rv | | CIP104536 |
| DBO | H37rv | ΔblaC | CIP104536 | Δbla_{Mab} |
| None | 128 | 1 | >256 | 4 |
| 1a.i.Na (15 μM) | 16 | 1 | 16 | 4 |

The results show that our synthetic DBO, 1a.i.Na penetrates into the periplasm of mycobacteria since it prevents the hydrolysis of amoxicillin by BlaC in M. tuberculosis (reduction in the MIC of amoxicillin from 128 μg/ml to 16 μg/ml) and by Bla_{Mab} in M. abscessus (reduction in the MIC of amoxicillin from >256 μg/ml to 16 μg/ml). In the absence of BlaC or Bla_{Mab} (deletion (Δ) of the β-lactamase gene blaC in H37Rv and blaMab in CIP104536), the MICs of amoxicillin were lower both in M. tuberculosis and M. abscessus, indicating that inhibition of the 13-lactamases BlaC and Bla_{Mab} is partial.

Together, these results indicate that the triazole ring generated by our biosynthetic route is compatible with (i) drug stability in the assay conditions, (ii) penetration into the periplasm, and (iii) β-lactamase inhibition.

Other results obtained are presented in Table 2, Table 3, Table 4 and Table 5.

TABLE 2
Potentiation of the activity of amoxicillin by diazabicyclooctanes^{a}

| | MIC of amoxicillin (μg/ml) against indicated strain | | | |
|---|---|---|---|---|
| | M. abscessus | | M. tuberculosis | |
| | | CIP104536 | | H37Rv |
| Inhibitor | CIP104536 | Δbla_{Mab} | H37Rv | ΔblaC |
| None | >256 | 4 | 128 | 1 |
| Avibactam | 16 | 4 | 8 | 0.5 |
| 1a.i | 16 | 4 | 16 | 1 |
| 1a.i.11 | 64 | 8 | 32 | 1 |
| 2a.i | 256 | 16 | 64 | 1 |
| 1a.i.13 | 256 | 8 | 128 | 0.5 |
| 1a.i.14 | >256 | 4 | 256 | 2 |
| 1a.i.16 | 64 | 8 | 32 | 0.5 |

DBOs were used at a concentration of 15 μM.

TABLE 3
Carbamoylation efficacy (M⁻¹ s⁻¹) of β-lactamases
BlaC and Bla_{Mab} by diazabicyclooctanes

| Inhibitor | Bla_{Mab} | BlaC |
|---|---|---|
| Avibactam | (1.7 ± 0.1) × 10⁵ | (2.4 ± 0.8) × 10¹ |
| 1a.i | (2.2 ± 0.1) × 10⁴ | <5 × 10⁰ |
| 1a.i.11 | (4.4 ± 0.2) × 10³ | <5 × 10⁰ |
| 2a.i | (6.2 ± 0.2) × 10⁴ | <5 × 10⁰ |
| 1a.i.13 | (2.0 ± 0.1) × 10³ | <5 × 10⁰ |
| 1a.i.14 | (1.8 ± 0.2) × 10³ | <5 × 10⁰ |
| 1a.i.16 | (2.3 ± 0.1) × 10⁵ | <5 × 10⁰ |

TABLE 4
Residual activity (%) of Ltd_{fm} following pre-incubation with diazabicyclooctanes

| Inhibitor/Ldt_{fm} | Inhibitor | | | | | | |
|---|---|---|---|---|---|---|---|
| molar ratio | Avibactam | 1a.i | 1a.i.11 | 2a.i | 1a.i.13 | 1a.i.14 | 1a.i.16 |
| 0 | 100 | 100 | 100 | 100 | 100 | 100 | 100 |
| 1 | 66.3 | 88.6 | 92.0 | 88.8 | 93.9 | 100.7 | 74.8 |
| 2.5 | 36.4 | 76.1 | 83.9 | 82.7 | 81.5 | 97.1 | 47.0 |
| 5 | 15.8 | 63.6 | 68.6 | 68.0 | 65.2 | 92.0 | 22.6 |
| 10 | 3.4 | 34.3 | 51.8 | 50.7 | 40.9 | 79.2 | 6.0 |
| 20 | 2.4 | 15.8 | 28.1 | 27.2 | 16.9 | 65.3 | 1.7 |

TABLE 5
Residual activity (%) of Ldt_{Mt2} following pre-incubation with diazabicyclooctanes

| Inhibitor/Ldt_{Mt2} | Inhibitor | | | | | | |
|---|---|---|---|---|---|---|---|
| molar ratio | Avibactam | 1a.i | 1a.i.11 | 2a.i | 1a.i.13 | 1a.i.14 | 1a.i.16 |
| 0 | 100 | 100 | 100 | 100 | 100 | 100 | 100 |
| 1 | 58.1 | 89.5 | 94.0 | 94.9 | 92.3 | 94.7 | 82.8 |
| 2.5 | 19.8 | 83.1 | 91.7 | 89.8 | 85.4 | 93.8 | 69.9 |
| 5 | 4.1 | 66.3 | 89.6 | 84.8 | 78.8 | 93.5 | 50.0 |
| 10 | 1.1 | 47.0 | 84.5 | 81.9 | 53.4 | 91.3 | 29.0 |
| 20 | 1.1 | 14.5 | 73.9 | 72.4 | 41.7 | 87.1 | 8.3 |

The biological activity of diazabiclyooctanes (DBOs) was evaluated by determining their ability to potentiate the activity of amoxicillin against mycobacteria (Table 2). The presence of avibactam caused a reduction of at least 16 fold in the minimal inhibitory concentrations (MICs) of amoxicillin against M. tuberculosis and M. abscessus. Inhibition of BlaC and Bla_{Mab} by avibactam was apparently partial since the MICs of amoxicillin remained higher than those observed for isogenic strains obtained by deletion of the blaC and blaMab genes. 1 a.i was the only other DBO that had an activity similar to that of avibactam, as estimated by the fold reduction in the MIC of amoxicillin. The other compounds were less active (e.g. 1a.i.11 and 1a.i.16).

Inhibition of the β-lactamase activity of BlaC and BlaMab was determined by using the chromogenic cephalosporin nitrocefin as the substrate (Table 3). The most efficacious inhibition of BlaMab was observed with avibactam and 1a.i.16. BlaC was poorly inhibited by avibactam and no inhibition was obtained with the other DBOs (tested up to 100 μM). This is in marked contrast with potentiation of the antibacterial activity of amoxicillin by certain DBOs against M. tuberculosis (Table 2). This observation suggests that the DBOs might not only inhibit the β-lactamases, but additionally act on peptidoglycan polymerases.

Inventors also investigated the inhibition of L,D-transpeptidases (LDTs), which are the main peptidoglycan cross-linking enzymes in mycobacteria. Inventors measured the residual rate of nitrocefin hydrolysis after pre-incubating Ldtₜₘ, a model LDT from E. faecium, and Ldt_{Mt2}, the main LDT of M. tuberculosis, with DBOs. Avibactam (200 μM) almost completely inhibited Ldtₜₘ and Ldt_{Mt2} (10 μM) (residual activity <3%). 1a.i.16 and avibactam were similarly active against both enzymes whereas inhibition of the L,D-transpeptidases with the other compounds was partial.

In conclusion, some of the synthetic DBOs were active in terms of target inhibition (Tables 4 and 5), β-lactamase inhibition (Table 3), and/or antibacterial activity in combination with amoxicillin (Table 2).

1. A compound of the following general formula (I): 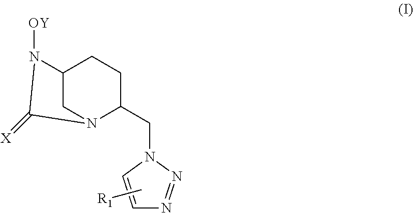
or a pharmaceutically acceptable salt and/or solvate thereof, wherein:
X is O or S;
Y is SO₃H or PO₃H; and
R₁ is:
H
a tri-(C₁-C₆)alkylsilyl group,
a (C₁-C₆)alkyl group, optionally substituted with one or several groups selected from halo, cyano (CN), OR₂, SR₃, NR₄R₅, COR₆, CO₂R₇, CONR₈R₉ and NO₂, or
an aryl, heteroaryl, aryl-(C₁-C₆)alkyl, heteroaryl-(C₁-C₆)alkyl, cycloalkyl, cycloalkyl-(C₁-C₆)alkyl, heterocycle or heterocycle-(C₁-C₆)alkyl group, optionally substituted with one or several groups selected from halo, cyano (CN), (C-C₆)alkyl, OR₁₀, SR₁₁, NR₁₂R₁₃, CORH₁₄, CO₂R₁₅, CONR₁₆R₁₇ and NO₂,
wherein R₂ to R₁₇ are, independently of each other, H, a (C₁-C₆)alkyl group or a C(═O)O(C₁-C₆)alkyl.

2. The compound according to claim 1, wherein said compound is of the following general formula (Ia):

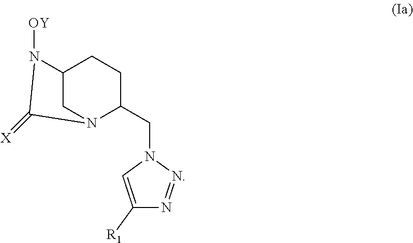

3. The compound according to claim 1 or claim 2, wherein X is O.

4. The compound according to claim 1, wherein Y is SO₃H.

5. The compound according to claim 1, wherein R₁ is:
a tri-(C₁-C₆)alkylsilyl group, notably a trimethylsilyl group,
a (C₁-C₆)alkyl group, optionally substituted with one or several groups selected from halo, OR₂, NR₄R₅, COR₇ and CONR₈R₉, or
an aryl, heteroaryl, aryl-(C₁-C₆)alkyl, heteroaryl-(C₁-C₆)alkyl, heterocycle or heterocycle-(C₁-C₆)alkyl group, optionally substituted with one or several groups selected from halo, (C₁-C₆)alkyl, OR₁₀, NR₁₂R₁₃, CO2Ris and CONR₁₆R₁₇.

6. The compound according to claim 1, wherein:
the aryl moiety in the aryl and aryl-(C₁-C₆)alkyl groups is a phenyl;
the heteroaryl moiety in the heteroaryl and heteroaryl-(C₁-C₆)alkyl groups is a 5- or 6- membered heteroaryl comprising one or two heteroatoms chosen from O and N, preferably selected from furan, pyrrole, imidazole, pyridine, pyrazine and pyrimidine, more preferably pyridine;
the heterocycle moiety in the heterocycle and heterocycle-(C₁l-C₆)alkyl groups is a 5- or 6- membered, saturated or unsaturated, preferably saturated heterocycle comprising one or two heteroatoms chosen from O and N, preferably selected from pyrrolidine, piperidine, morpholine and piperazine.

7. The compound according to claim 1, wherein it is chosen among the following compounds 1a.i and 2a.i: 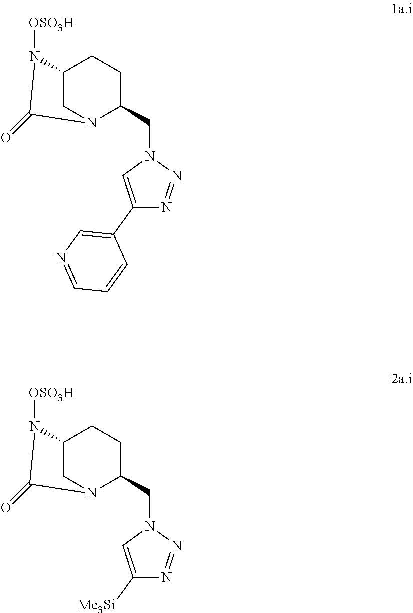
and the pharmaceutically acceptable salts, such as the sodium salts, and solvates thereof.

8. A compound according to claim 1 for use as a drug.

9. A compound according to claim 1 for use as a β-lactamase inhibitor.

10. A compound according to claim 1 for use in the treatment of a disease caused by mycobacteria, in particular M. tuberculosis or M. abscessus.

11. A pharmaceutical composition comprising at least one compound according to claim 1 and at least one pharmaceutically acceptable excipient.

12. A pharmaceutical composition comprising:
(i) at least one compound according to claim 1, and
(ii) at least another active principle, such as an antibiotic, notably a β-lactam antibiotic, as a combination product for a simultaneous, separate or sequential use.

13. A process to prepare the compound according to claim 1, comprising a reaction converting the OH group of a compound of the following formula (II) into a OY group to obtain the corresponding compound of formula (I): 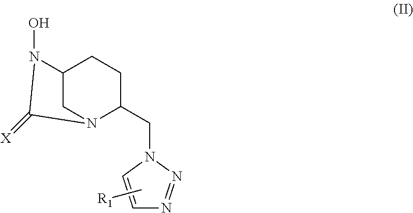
wherein X is O or S, and R₁ is as defined in claim 1, R₁ being optionally in a protected form, wherein:
when Y is SO₃H, said reaction is a sulfonation reaction, and
when Y is PO₃H, said reaction is a phosphorylation reaction,
followed by a deprotection of the R₁ group when it is in a protected form,
optionally followed by a salt-forming step.

14. The process according to claim 13, wherein the compound of formula (II) is obtained by a coupling reaction between:
a compound of the following formula (III): 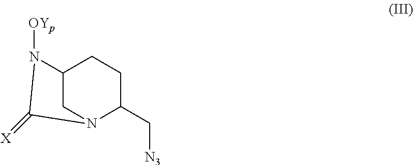
wherein X is O or S, and Yₚ is a hydroxyl protecting group, such as a benzyl group, and
a compound of the following formula (IV): 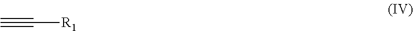
wherein R₁ is as defined in claim 1, optionally in a protected form,
followed by a deprotection of the OYₚ group.
